SEQUENCE LISTING (35 sequences)

SEQ ID NO: 1 - protein, 208 aa, Homo sapiens
MPPYTVVYFPVRGRCAALRMLLADQGQSWKEEVVTVETWQESLKASCLYGQLPKFQDGDLTLYQSNTILRHLGRTLGLYGKDQQEAALDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 2 - protein, 585 aa, Homo sapiens [sequence omitted]

SEQ ID NO: 3 - protein, 624 aa, Homo sapiens
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 4 - protein, 959 aa, Homo sapiens
MAASTGYVRLWGAARCWVLRRPMLAAAGGRVPTAAGAWLLRGQRTCDASPPWALWGRGPAIGGQWRGFWEASSRGGGAFSGGEDASEGGAEEGAGGAGGSAGAGEGPVITALTPMTIPDVFPHLPLIAITRNPVFPRFIKIIEVKNKKLVELLRRKVRLAQPYVGVFLKRDDSNESDVVESLDEIYHTGTFAQIHEMQDLGDKLRMIVMGHRRVHISRQLEVEPEEPEAENKHKPRRKSKRGKKEAEDELSARHPAELAMEPTPELPAEVLMVEVENVVHEDFQVTEEVKALTAEIVKTIRDIIALNPLYRESVLQMMQAGQRVVDNPIYLSDMGAALTGAESHELQDVLEETNIPKRLYKALSLLKKEFELSKLQQRLGREVEEKIKQTHRKYLLQEQLKIIKKELGLEKDDKDAIEEKFRERLKELVVPKHVMDVVDEELSKLGLLDNHSSEFNVTRNYLDWLTSIPWGKYSNENLDLARAQAVLEEDHYGMEDVKKRILEFIAVSQLRGSTQGKILCFYGPPGVGKTSIARSIARALNREYFRFSVGGMTDVAEIKGHRRTYVGAMPGKIIQCLKKTKTENPLILIDEVDKIGRGYQGDPSSALLELLDPEQNANFLDHYLDVPVDLSKVLFICTANVTDTIPEPLRDRMEMINVSGYVAQEKLAIAERYLVPQARALCGLDESKAKLSSDVLTLLIKQYCRESGVRNLQKQVEKVLRKSAYKIVSGEAESVEVTPENLQDFVGKPVFTVERMYDVTPPGVVMGLAWTAMGGSTLFVETSLRRPQDKDAKGDKDGSLEVTGQLGEVMKESARIAYTFARAFLMQHAPANDYLVTSHIHLHVPEGATPKDGPSAGCTIVTALLSLAMGRPVRQNLAMTGEVSLTGKILPVGGIKEKTIAAKRAGVTCIVLPAENKKDFYDLAAFITEGLEVHFVEHYREIFDIAFPDEQAEALAVER

SEQ ID NO: 5 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 6 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 7 - protein, 704 aa, Homo sapiens
MAEAAEPEGVAPGPQGPPEVPAPLAERPGEPGAAGGEAEGPEGSEGAEEAPRGAAAVKEAGGGGPDRGPEAEARGTRGAHGETEAEEGAPEGAEVPQGGEETSGAQQVEGASPGRGAQGEPRGEAQREPEDSAAPERQEEAEQRPEVPEGSASGEAGDSVDAEGPLGDNIEAEGPAGDSVEAEGRVGDSVDAEGPAGDSVDAEGPLGDNIQAEGPAGDSVDAEGRVGDSVDAEGPAGDSVDAEGRVGDSVEAGDPAGDGVEAGVPAGDSVEAEGPAGDSMDAEGPAGRARRVSGEPQQSGDGSLSPQAEAIEVAAGESAGRSPGELAWDAAEEAEVPGVKGSEEAAPGDARADAGEDRVGDGPQQEPGEDEERRERSPEGPREEEAAGGEEESPDSSPHGEASRGAAEPEAQLSNHLAEEGPAEGSGEAARVNGRREDGEASEPRALGQEHDITLFVKVKLTALGCSRIAIKKYLRAGYDGESIGNCPFSQRLFMILWLKGVIFNVTTVDLKRKPADLQNLAPGTNPPFMTFDGEVKTDVNKIEEFLEEKLAPPRYPKLGTQHPESNSAGNDVFAKFSAFIKNTKKDANEIHEKNLLKALRKLDNYLNSPLPDEIDAYSTEDVTVSGRKFLDGDELTLADCNLLPKLHIIKIVAKKYRDFEFPSEMTGIWRYLNNAYARDEFTNTCPADQEIEHAYSDVAKRMK

SEQ ID NO: 8 - protein, 2588 aa, Homo sapiens
MGSPEGRFHFAIDRGGTFTDVFAQCPGGHVRVLKLLSEDPANYADAPTEGIRRILEQEAGMLLPRDQPLDSSHIASIRMGTTVATNALLERKGERVALLVTRGFRDLLHIGTQARGDLFDLAVPMPEVLYEEVLEVDERVVLHRGEAGTGTPVKGRTGDLLEVQQPVDLGALRGKLEGLLSRGIRSLAVVLMHSYTWAQHEQQVGVLARELGFTHVSLSSEAMPMVRIVPRGHTACADAYLTPAIQRYVQGFCRGFQGQLKDVQVLFMRSDGGLAPMDTFSGSSAVLSGPAGGVVGYSATTYQQEGGQPVIGFDMGGTSTDVSRYAGEFEHVFEASTAGVTLQAPQLDINTVAAGGGSRLFFRSGLFVVGPESAGAHPGPACYRKGGPVTVTDANLVLGRLLPASFPCIFGPGENQPLSPEASRKALEAVATEVNSFLTNGPCPASPLSLEEVAMGFVRVANEAMCRPIRALTQARGHDPSAHVLACFGGAGGQHACAIARALGMDTVHIHRHSGLLSALGLALADVVHEAQEPCSLLYAPETFVQLDQRLSRLEEQCVDALQAQGFPRSQISTESFLHLRYQGTDCALMVSAHQHPATARSPRAGDFGAAFVERYMREFGFVIPERPVVVDDVRVRGTGRSGLRLEDAPKAQTGPPRVDKMTQCYFEGGYQETPVYLLAELGYGHKLHGPCLIIDSNSTILVEPGCQAEVTKTGDICISVGAEVPGTVGPQLDPIQLSIFSHRFMSIAEQMGRILQRTAISTNIKERLDFSCALFGPDGGLVSNAPHIPVHLGAMQETVQFQIQHLGADLHPGDVLLSNHPSAGGSHLPDLTVITPVFWPGQTRPVFYVASRGHHADIGGITPGSMPPHSTMLQQEGAVFLSFKLVQGGVFQEEAVTEALRAPGKVPNCSGTRNLHDNLSDLRAQVAANQKGIQLVGELIGQYGLDVVQAYMGHIQANAELAVRDMLRAFGTSRQARGLPLEVSSEDHMDDGSPIRLRVQISLSQGSAVFDFSGTGPEVFGNLNAPRAVTLSALIYCLRCLVGRDIPLNQGCLAPVRVVIPRGSILDPSPEAAVVGGNVLTSQRVVDVILGAFGACAASQGCMNNVTLGNAHMGYYETVAGGAGAGPSWHGRSGVHSHMTNTRITDPEILESRYPVILRRFELRRGSGGRGRFRGGDGVTRELLFREEALLSVLTERRAFRPYGLHGGEPGARGLNLLIRKNGRTVNLGGKTSVTVYPGDVFCLHTPGGGGYGDPEDPAPPPGSPPQALAFPEHGSVYEYRRAQEAVMGTGPAQTPRSTRAGPEPSPAPPGPGDTGDSDVTQEGSGPAGIRGGETVIRAGMGDSPGRGAPERRHKAQPGRARKYEWRPEGPTSMGSLGQREDLQDEDRNSAFTWKVQANNRAYNGQFKEKVILCWQRKKYKTNVIRTAKYNFYSFLPLNLYEQFHRVSNLFFLIIIILQSIPDISTLPWFSLSTPMVCLLFIRATRDLVDDMGRHKSDRAINNRPCQILMGKSFKQKKWQDLCVGDVVCLRKDNIVPADMLLLASTEPSSLCYVETVDIDGETNLKFRQALMVTHKELATIKKMASFQGTVTCEAPNSRMHHFVGCLEWNDKKYSLDIGNLLLRGCRIRNTDTCYGLVIYAGFDTKIMKNCGKIHLKRTKLDLLMNKLVVVIFISVVLVCLVLAFGFGFSVKEFKDHHYYLSGVHGSSVAAESFFVFWSFLILLSVTIPMSMFILSEFIYLGNSVFIDWDVQMYYKPQDVPAKARSTSLNDHLGQVEYIFSDKTGTLTQNILTFNKCCISGRVYGPDSEATTRPKENPYLWNKFADGKLLFHNAALLHLVRTNGDEAVREFWRLLAICHTVMVRESPRERPDQLLYQAASPDEGALVTAARNFGYVFLSRTQDTVTIMELGEERVYQVLAIMDFNSTRKRMSVLVRKPEGAICLYTKGADTVIFERLHRRGAMEFATEEALAAFAQETLRTLCLAYREVAEDIYEDWQQRHQEASLLLQNRAQALQQLLGATAIEDRLQDGVPETIKCLKKSNIKIWVLTGDKQETAVNIGFACELLSENMLILEEKEISRILETYWENSNNLLTRESLSQVKLALVINGDFLDKLLVSLRKEPRALAQNVNMDEAWQELGQSRRDFLYARRLSLLCRRFGLPLAAPPAQDSRARRSSEVLQERAFVDLASKCQAVICCRVTPKQKALIVALVKKYHQVVTLAIGDGANDINMIKTADVGVGLAGQEGMQAVQNSDFVLGQFCFLQRLLLVHGRWSYVRICKFLRYFFYKSMASMMVQVWFACYNGFTGQPLYEGWFLALFNLLYSTLPVLYIGLFEQDVSAEQSLEKPELYVVGQKDELFNYWVFVQAIAHGVTTSLVNFFMTLWISRDTAGPASFSDHQSFAVVVALSCLLSITMEVILIIKYWTALCVATILLSLGFYAIMTTTTQSFWLFRVSPTTFPFLYADLSVMSSPSILLVVLLSVSINTFPVLALRVIFPALKELRAKEEKVEEGPSEEIFTMEPLPHVHRESRARRSSYAFSHREGYANLITQGTILRRGPGVSSDIASESLDPSDEEAASSPKESQ

SEQ ID NO: 9 - protein, 1300 aa, Homo sapiens
MGTGPAQTPRSTRAGPEPSPAPPGPGDTGDSDVTQEGSGPAGIRGGETVIRAGMGDSPGRGAPERRHKAQPGRARKYEWRPEGPTSMGSLGQREDLQDEDRNSAFTWKVQANNRAYNGQFKEKVILCWQRKKYKTNVIRTAKYNFYSFLPLNLYEQFHRVSNLFFLIIIILQSIPDISTLPWFSLSTPMVCLLFIRATRDLVDDMGRHKSDRAINNRPCQILMGKSFKQKKWQDLCVGDVVCLRKDNIVPADMLLLASTEPSSLCYVETVDIDGETNLKFRQALMVTHKELATIKKMASFQGTVTCEAPNSRMHHFVGCLEWNDKKYSLDIGNLLLRGCRIRNTDTCYGLVIYAGFDTKIMKNCGKIHLKRTKLDLLMNKLVVVIFISVVLVCLVLAFGFGFSVKEFKDHHYYLSGVHGSSVAAESFFVFWSFLILLSVTIPMSMFILSEFIYLGNSVFIDWDVQMYYKPQDVPAKARSTSLNDHLGQVEYIFSDKTGTLTQNILTFNKCCISGRVYGPDSEATTRPKENPYLWNKFADGKLLFHNAALLHLVRTNGDEAVREFWRLLAICHTVMVRESPRERPDQLLYQAASPDEGALVTAARNFGYVFLSRTQDTVTIMELGEERVYQVLAIMDFNSTRKRMSVLVRKPEGAICLYTKGADTVIFERLHRRGAMEFATEEALAAFAQETLRTLCLAYREVAEDIYEDWQQRHQEASLLLQNRAQALQQLLGATAIEDRLQDGVPETIKCLKKSNIKIWVLTGDKQETAVNIGFACELLSENMLILEEKEISRILETYWENSNNLLTRESLSQVKLALVINGDFLDKLLVSLRKEPRALAQNVNMDEAWQELGQSRRDFLYARRLSLLCRRFGLPLAAPPAQDSRARRSSEVLQERAFVDLASKCQAVICCRVTPKQKALIVALVKKYHQVVTLAIGDGANDINMIKTADVGVGLAGQEGMQAVQNSDFVLGQFCFLQRLLLVHGRWSYVRICKFLRYFFYKSMASMMVQVWFACYNGFTGQPLYEGWFLALFNLLYSTLPVLYIGLFEQDVSAEQSLEKPELYVVGQKDELFNYWVFVQAIAHGVTTSLVNFFMTLWISRDTAGPASFSDHQSFAVVVALSCLLSITMEVILIIKYWTALCVATILLSLGFYAIMTTTTQSFWLFRVSPTTFPFLYADLSVMSSPSILLVVLLSVSINTFPVLALRVIFPALKELRAKEEKVEEGPSEEIFTMEPLPHVHRESRARRSSYAFSHREGYANLITQGTILRRGPGVSSDIASESLDPSDEEAASSPKESQ

SEQ ID NO: 10 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 11 - protein, 444 aa, Homo sapiens
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 12 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 13 - protein, 295 aa, Homo sapiens [sequence omitted]

SEQ ID NO: 14 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 15 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 16 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

SEQ ID NO: 17 - protein, 1058 aa, Homo sapiens
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 18 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 19 - protein, 461 aa, Homo sapiens
MSRQVVRSSKFRHVFGQPAKADQCYEDVRVSQTTWDSGFCAVNPKFVALICEASGGGAFLVLPLGKTGRVDKNAPTVCGHTAPVLDIAWCPHNDNVIASGSEDCTVMVWEIPDGGLMLPLREPVVTLEGHTKRVGIVAWHTTAQNVLLSAGCDNVIMVWDVGTGAAMLTLGPEVHPDTIYSVDWSRDGGLICTSCRDKRVRIIEPRKGTVVAEKDRPHEGTRPVRAVFVSEGKILTTGFSRMSERQVALWDTKHLEEPLSLQELDTSSGVLLPFFDPDTNIVYLCGKGDSSIRYFEITSEAPFLHYLSMFSSKESQRGMGYMPKRGLEVNKCEIARFYKLHERRCEPIAMTVPRKSDLFQEDLYPPTAGPDPALTAEEWLGGRDAGPLLISLKDGYVPPKSRELRVNRGLDTGRRRAAPEASGTPSSDAVSRLEEEMRKLQATVQELQKRLDRLEETVQAK

SEQ ID NO: 20 - protein, 847 aa, Homo sapiens
MSKSFQQSSLSRDSQGHGRDLSAAGIGLLAAATQSLSMPASLGRMNQGTARLASLMNLGMSSSLNQQGAHSALSSASTSSHNLQSIFNIGSRGPLPLSSQHRGDADQASNILASFGLSARDLDELSRYPEDKITPENLPQILLQLKRRRTEEGPTLSYGRDGRSATREPPYRVPRDDWEEKRHFRRDSFDDRGPSLNPVLDYDHGSRSQESGYYDRMDYEDDRLRDGERCRDDSFFGETSHNYHKFDSEYERMGRGPGPLQERSLFEKKRGAPPSSNIEDFHGLLPKGYPHLCSICDLPVHSNKEWSQHINGASHSRRCQLLLEIYPEWNPDNDTGHTMGDPFMLQQSTNPAPGILGPPPPSFHLGGPAVGPRGNLGAGNGNLQGPRHMQKGRVETSRVVHIMDFQRGKNLRYQLLQLVEPFGVISNHLILNKINEAFIEMATTEDAQAAVDYYTTTPALVFGKPVRVHLSQKYKRIKKPEGKPDQKFDQKQELGRVIHLSNLPHSGYSDSAVLKLAEPYGKIKNYILMRMKSQAFIEMETREDAMAMVDHCLKKALWFQGRCVKVDLSEKYKKLVLRIPNRGIDLLKKDKSRKRSYSPDGKESPSDKKSKTDGSQKTESSTEGKEQEEKSGEDGEKDTKDDQTEQEPNMLLESEDELLVDEEEAAALLESGSSVGDETDLANLGDVASDGKKEPSDKAVKKDGSASAAAKKKLKKVDKIEELDQENEAALENGIKNEENTEPGAESSENADDPNKDTSENADGQSDENKDDYTIPDEYRIGPYQPNVPVGIDYVIPKTGFYCKLCSLFYTNEEVAKNTHCSSLPHYQKLKKFLNKLAEERRQKKET

SEQ ID NO: 21 - protein, 204 aa, Homo sapiens [sequence omitted]

SEQ ID NO: 22 - protein, 923 aa, Homo sapiens
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSFSDDGALGPVLTTFDHTLDHESLNPGAQRFEKMIGGLYLGELVRLVLAHLARCGVLFGGCTSPALLSQGSILLEHVAEMEDPSTGAARVHAILQDLGLSPGASDVELVQHVCAAVCTRAAQLCAAALAAVLSCLQHSREQQTLQVAVATGGRVCERHPRFCSVLQGTVMLLAPECDVSLIPSVDGGGRGVAMVTAVAARLAAHRRLLEETLAPFRLNHDQLAAVQAQMRKAMAKGLRGEASSLRMLPTFVRATPDGSERGDFLALDLGGTNFRVLLVRVTTGVQITSEIYSIPETVAQGSGQQLFDHIVDCIVDFQQKQGLSGQSLPLGFTFSFPCRQLGLDQGILLNWTKGFKASDCEGQDVVSLLREAITRRQAVELNVVAIVNDTVGTMMSCGYEDPRCEIGLIVGTGTNACYMEELRNVAGVPGDSGRMCINMEWGAFGDDGSLAMLSTRFDASVDQASINPGKQRFEKMISGMYLGEIVRHILLHLTSLGVLFRGQQIQRLQTRDIFKTKFLSEIESDSLALRQVRAILEDLGLPLTSDDALMVLEVCQAVSQRAAQLCGAGVAAVVEKIRENRGLEELAVSVGVDGTLYKLHPRFSSLVAATVRELAPRCVVTFLQSEDGSGKGAALVTAVACRLAQLTRV

SEQ ID NO: 23 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 24 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 25 - protein, 83 aa, Homo sapiens
MQNDAGEFVDLYVPRKCSASNRIIGAKDHASIQMNVAEVDKVTGRFNGQFKTYAICGAIRRMGESDDSILRLAKADGIVSKNF

SEQ ID NO: 26 - protein, 451 aa, Homo sapiens [sequence omitted]

SEQ ID NO: 27 - protein, 164 aa, Homo sapiens
MHRDSCPLDCKVYVGNLGNNGNKTELERAFGYYGPLRSVWVARNPPGFAFVEFEDPRDAADAVRELDGRTLCGCRVRVELSNGEKRSRNRGPPPSWGRRPRDDYRRRSPPPRRRSPRRRSFSRSRSRSLSRDRRRERSLSRERNHKPSRSFSRSRSRSRSNERK

SEQ ID NO: 28 - protein, 825 aa, Homo sapiens
MSSSPVNVKKLKVSELKEELKKRRLSDKGLKAELMERLQAALDDEEAGGRPAMEPGNGSLDLGGDSAGRSGAGLEQEAAAGGDEEEEEEEEEEEGISALDGDQMELGEENGAAGAADSGPMEEEEAASEDENGDDQGFQEGEDELGDEEEGAGDENGHGEQQPQPPATQQQQPQQQRGAAKEAAGKSSGPTSLFAVTVAPPGARQGQQQAGGKKKAEGGGGGGRPGAPAAGDGKTEQKGGDKKRGVKRPREDHGRGYFEYIEENKYSRAKSPQPPVEEEDEHFDDTVVCLDTYNCDLHFKISRDRLSASSLTMESFAFLWAGGRASYGVSKGKVCFEMKVTEKIPVRHLYTKDIDIHEVRIGWSLTTSGMLLGEEEFSYGYSLKGIKTCNCETEDYGEKFDENDVITCFANFESDEVELSYAKNGQDLGVAFKISKEVLAGRPLFPHVLCHNCAVEFNFGQKEKPYFPIPEEYTFIQNVPLEDRVRGPKGPEEKKDCEVVMMIGLPGAGKTTWVTKHAAENPGKYNILGTNTIMDKMMVAGFKKQMADTGKLNTLLQRAPQCLGKFIEIAARKKRNFILDQTNVSAAAQRRKMCLFAGFQRKAVVVCPKDEDYKQRTQKKAEVEGKDLPEHAVLKMKGNFTLPEVAECFDEITYVELQKEEAQKLLEQYKEESKKALPPEKKQNTGSKKSNKNKSGKNQFNRGGGHRGRGGFNMRGGNFRGGAPGNRGGYNRRGNMPQRGGGGGGSGGIGYPYPRAPVFPGRGSYSNRGNYNRGGMPNRGNYNQNFRGRGNNRGYKNQSQGYNQWQQGQFWGQKPWSQHYHQGYY

SEQ ID NO: 29 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 30 - protein, 2813 aa, Homo sapiens
MKLNPQQAPLYGDCVVTVLLAEEDKAEDDVVFYLVFLGSTLRHCTSTRKVSSDTLETIAPGHDCCETVKVQLCASKEGLPVFVVAEEDFHFVQDEAYDAAQFLATSAGNQQALNFTRFLDQSGPPSGDVNSLDKKLVLAFRHLKLPTEWNVLGTDQSLHDAGPRETLMHFAVRLGLLRLTWFLLQKPGGRGALSIHNQEGATPVSLALERGYHKLHQLLTEENAGEPDSWSSLSYEIPYGDCSVRHHRELDIYTLTSESDSHHEHPFPGDGCTGPIFKLMNIQQQLMKTNLKQMDSLMPLMMTAQDPSSAPETDGQFLPCAPEPTDPQRLSSSEETESTQCCPGSPVAQTESPCDLSSIVEEENTDRSCRKKNKGVERKGEEVEPAPIVDSGTVSDQDSCLQSLPDCGVKGTEGLSSCGNRNEETGTKSSGMPTDQESLSSGDAVLQRDLVMEPGTAQYSSGGELGGISTTNVSTPDTAGEMEHGLMNPDATVWKNVLQGGESTKERFENSNIGTAGASDVHVTSKPVDKISVPNCAPAASSLDGNKPAESSLAFSNEETSTEKTAETETSRSREESADAPVDQNSVVIPAAAKDKISDGLEPYTLLAAGIGEAMSPSDLALLGLEEDVMPHQNSETNSSHAQSQKGKSSPICSTTGDDKLCADSACQQNTVTSSGDLVAKLCDNIVSESESTTARQPSSQDPPDASHCEDPQAHTVTSDPVRDTQERADFCPFKVVDNKGQRKDVKLDKPLTNMLEVVSHPHPVVPKMEKELVPDQAVISDSTFSLANSPGSESVTKDDALSFVPSQKEKGTATPELHTATDYRDGPDGNSNEPDTRPLEDRAVGLSTSSTAAELQHGMGNTSLTGLGGEHEGPAPPAIPEALNIKGNTDSSLQSVGKATLALDSVLTEEGKLLVVSESSAAQEQDKDKAVTCSSIKENALSSGTLQEEQRTPPPGQDTQQFHEKSISADCAKDKALQLSNSPGASSAFLKAETEHNKEVAPQVSLLTQGGAAQSLVPPGASLATESRQEALGAEHNSSALLPCLLPDGSDGSDALNCSQPSPLDVGVKNTQSQGKTSACEVSGDVTVDVTGVNALQGMAEPRRENISHNTQDILIPNVLLSQEKNAVLGLPVALQDKAVTDPQGVGTPEMIPLDWEKGKLEGADHSCTMGDAEEAQIDDEAHPVLLQPVAKELPTDMELSAHDDGAPAGVREVMRAPPSGRERSTPSLPCMVSAQDAPLPKGADLIEEAASRIVDAVIEQVKAAGALLTEGEACHMSLSSPELGPLTKGLESAFTEKVSTFPPGESLPMGSTPEEATGSLAGCFAGREEPEKIILPVQGPEPAAEMPDVKAEDEVDFRASSISEEVAVGSIAATLKMKQGPMTQAINRENWCTIEPCPDAASLLASKQSPECENFLDVGLGRECTSKQGVLKRESGSDSDLFHSPSDDMDSIIFPKPEEEHLACDITGSSSSTDDTASLDRHSSHGSDVSLSQILKPNRSRDRQSLDGFYSHGMGAEGRESESEPADPGDVEEEEMDSITEVPANCSVLRSSMRSLSPFRRHSWGPGKNAASDAEMNHRSSMRVLGDVVRRPPIHRRSFSLEGLTGGAGVGNKPSSSLEVSSANAEELRHPFSGEERVDSLVSLSEEDLESDQREHRMFDQQICHRSKQQGFNYCTSAISSPLTKSISLMTISHPGLDNSRPFHSTFHNTSANLTESITEENYNFLPHSPSKKDSEWKSGTKVSRTFSYIKNKMSSSKKSKEKEKEKDKIKEKEKDSKDKEKDKKTVNGHTFSSIPVVGPISCSQCMKPFTNKDAYTCANCSAFVHKGCRESLASCAKVKMKQPKGSLQAHDTSSLPTVIMRNKPSQPKERPRSAVLLVDETATTPIFANRRSQQSVSLSKSVSIQNITGVGNDENMSNTWKFLSHSTDSLNKISKVNESTESLTDEGVGTDMNEGQLLGDFEIESKQLEAESWSRIIDSKFLKQQKKDVVKRQEVIYELMQTEFHHVRTLKIMSGVYSQGMMADLLFEQQMVEKLFPCLDELISIHSQFFQRILERKKESLVDKSEKNFLIKRIGDVLVNQFSGENAERLKKTYGKFCGQHNQSVNYFKDLYAKDKRFQAFVKKKMSSSVVRRLGIPECILLVTQRITKYPVLFQRILQCTKDNEVEQEDLAQSLSLVKDVIGAVDSKVASYEKKVRLNEIYTKTDSKSIMRMKSGQMFAKEDLKRKKLVRDGSVFLKNAAGRLKEVQAVLLTDILVFLQEKDQKYIFASLDQKSTVISLKKLIVREVAHEEKGLFLISMGMTDPEMVEVHASSKEERNSWIQIIQDTINTLNRDEDEGIPSENEEEKKMLDTRARELKEQLHQKDQKILLLLEEKEMIFRDMAECSTPLPEDCSPTHSPRVLFRSNTEEALKGGPLMKSAINEVEILQGLVSGNLGGTLGPTVSSPIEQDVVGPVSLPRRAETFGGFDSHQMNASKGGEKEEGDDGQDLRRTESDSGLKKGGNANLVFMLKRNSEQVVQSVVHLYELLSALQGVVLQQDSYIEDQKLVLSERALTRSLSRPSSLIEQEKQRSLEKQRQDLANLQKQQAQYLEEKRRREREWEARERELREREALLAQREEEVQQGQQDLEKEREELQQKKGTYQYDLERLRAAQKQLEREQEQLRREAERLSQRQTERDLCQVSHPHTKLMRIPSFFPSPEEPPSPSAPSIAKSGSLDSELSVSPKRNSISRTHKDKGPFHILSSTSQTNKGPEGQSQAPASTSASTRLFGLTKPKEKKEKKKKNKTSRSQPGDGPASEVSAEGEEIFC

SEQ ID NO: 31 - protein, 356 aa, Homo sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 32 - protein, 270 aa, Homo sapiens
PWSECSATCAGGVQRQEVVCKRLDDNSIVQNNYCDPDSKPPENQRACNTEPCPPEWFIGDWLECSKTCDGGMRTRAVLCIRKIGPSEEETLDYSGCLTHRPVEKEPCNNQSCPPQWVALDWSECTPKCGPGFKHRIVLCKSSDLSKTFPAAQCPEESKPPVRIRCSLGRCPPPRWVTGDWGQCSAQCGLGQQMRTVQCLSYTGQASSDCLETVRPPSMQQCESKCDSTPISNTEECKDVNKVAYCPLVLKFKFCSRAYFRQMCCKTCQGH

SEQ ID NO: 33 - protein, 1560 aa, Homo sapiens
MQPGSAPPPGRMDPSAPQPRAETSGKDIWHPGERCLAPSPDNGKLCEASIKSITVDENGKSFAVVLYADFQERKIPLKQLQEVKFVKDCPRNLIFDDEDLEKPYFPNRKFPSSSVAFKLSDNGDSIPYTINRYLRDYQREGTRFLYGHYIHGGGCILGDDMGLGKTVQVISFLAAVLHKKGTREDIENNMPEFLLRSMKKEPLSSTAKKMFLIVAPLSVLYNWKDELDTWGYFRVTVLHGNRKDNELIRVKQRKCEIALTTYETLRLCLDELNSLEWSAVIVDEAHRIKNPKARVTEVMKALKCNVRIGLTGTILQNNMKELWCVMDWAVPGLLGSGTYFKKQFSDPVEHGQRHTATKRELATGRKAMQRLAKKMSGWFLRRTKTLIKDQLPKKEDRMVYCSLTDFQKAVYQTVLETEDVTLILQSSEPCTCRSGQKRRNCCYKTNSHGETVKTLYLSYLTVLQKVANHVALLQAASTSKQQETLIKRICDQVFSRFPDFVQKSKDAAFETLSDPKYSGKMKVLQQLLNHCRKNRDKVLLFSFSTKLLDVLQQYCMASGLDYRRLDGSTKSEERLKIVKEFNSTQDVNICLVSTMAGGLGLNFVGANVVVLFDPTWNPANDLQAIDRAYRIGQCRDVKVLRLISLGTVEEIMYLRQIYKQQLHCVVVGSENAKRYFEAVQGSKEHQGELFGIHNLFKFRSQGSCLTKDILEREGQVEAGIMTATTWLKEGPPAHKLEMPRQPDCQECRGTEQAAEPLAKEACDLCSDFSDEEPVGATGIKTAKNKAPDSSKASSSPGQLTLLQCGFSKLLETKCKAVEDSDGNTASDDESSDEQPTCLSTEAKDAGCEKNQDSLGTSKHQKLDNILNPKEKHIFYKSEKILEQNISSKSDEKKIKNTDKHCILQNVTESEDSDVICPTQYTTERFPDNSIRFKPPLEGSEDSETEHTVKTRNNDNSRNTDDKRNGIISKKLSPENTTLKSILKRKGTSDISDESDDIEISSKSRVRKRASSLRFKRIKETKKELHNSPKTMNKTNQVYAANEDHNSQFIDDYSSSDESLSVSHFSFSKQSHRPRTIRDRTSFSSKLPSHNKKNSTFIPRKPMKCSNEKVVNQEQSYESMDKFLDGVQEVAYIHSNQNVIGSSKAENHMSRWAAHDVFELKQFSQLPANIAVCSSKTYKEKVDADTLPHTKKGQQPSEGSISLPLYISNPVNQKKKKVYHTNQTTFIIGETPKGIRRKQFEEMASYFNSSSVNEFAKHITNATSEERQKMLRDFYASQYPEVKEFFVDSVSQFNNSSFEKGEQRTRKKSDKRESLIKPRLSDSETLSFKDSTNKISQVCSLKTYKRKSVKFQNHISYREEVFFNDAETKKSPVSSTQEIDSGKNSQASEDTVTSRSLNSESETRERRLENTMKDQQDLTRTGISRKEPLLKLENKKIENPVLENTSVISLLGDTSILDDLFKSHGNSPTQLPKKVLSGPMEKAKQRPKDFWDILNEQNDESLSKLTDLAVIETLCEKAPLAAPFKRREEPATSLWKSNEKFLWKKFSPSDTDENATNTQST

SEQ ID NO: 34 - protein, 711 aa, Homo sapiens
MSDYSTGGPPPGPPPPAGGGGGAGGAGGGPPPGPPGAGDRGGGGPGGGGPGGGSAGGPSQPPGGGGPGIRKDAFADAVQRARQIAAKIGGDAATTVNNSTPDFGFGGQKRQLEDGDQPESKKLASQGDSISSQLGPIHPPPRTSMTEEYRVPDGMVGLIIGRGGEQINKIQQDSGCKVQISPDSGGLPERSVSLTGAPESVQKAKMMLDDIVSRGRGGPPGQFHDNANGGQNGTVQEIMIPAGKAGLVIGKGGETIKQLQERAGVKMILIQDGSQNTNVDKPLRIIGDPYKVQQACEMVMDILRERDQGGFGDRNEYGSRIGGGIDVPVPRHSVGVVIGRSGEMIKKIQNDAGVRIQFKQDDGTGPEKIAHIMGPPDRCEHAARIINDLLQSLRSGPPGPPGGPGMPPGGRGRGRGQGNWGPPGGEMTFSIPTHKCGLVIGRGGENVKAINQQTGAFVEISRQLPPNGDPNFKLFIIRGSPQQIDHAKQLIEEKIEGPLCPVGPGPGGPGPAGPMGPFNPGPFNQGPPGAPPHAGGPPPHQYPPQGWGNTYPQWQPPAPHDPSKAAAAAADPNAAWAAYYSHYYQQPPGPVPGPAPAPAAPPAQGEPPQPPPTGQSDYTKAWEEYYKKIGQQPQQPGAPPQQDYTKAWEEYYKKQAQVATGGGPGAPPGSQPDYSAAWAEYYRQQAAYYGQTPGPGGPQPPPTQQGQQQAQ

SEQ ID NO: 35 - protein, 445 aa, Homo sapiens [sequence omitted]

(19) United States
(12) Patent Application Publication
(10) Pub. No.: US 2026/0191966 A1
(43) Pub. Date: Jul. 9, 2026

(54) METHODS AND COMPOSITIONS FOR IRREVERSIBLE ENZYME INHIBITION

(71) Applicant: TRITERPENOID THERAPEUTICS, INC., Lebanon, NH (US)

(72) Inventors: Michael B. SPORN, Tunbridge, VT (US); Xiaoli MENG, Lebanon, NH (US)

(21) Appl. No.: 19/462,670

(22) Filed: Jan. 28, 2026

Related U.S. Application Data
(63) Continuation of application No. 17/594,287, filed on Apr. 10, 2020, now Pat. No. 12,551,562.
(60) Provisional application No. 62/832,543, filed on Apr. 11, 2019.

(51) Int. Cl. A61K 47/54; A61P 43/00; C07J 63/00

(52) U.S. Cl. CPC A61K 47/545; A61P 43/00; C07J 63/008

(57) ABSTRACT

The present invention relates to compounds, methods, and compositions for irreversibly inhibiting protein targets, including Keap1 and/or LONP1 protease. In certain aspects, the present invention relates to conjugates and/or cross-linked conjugates comprising a protein or a protein complex and a bifunctional triterpenoid, such as 1-[2-Cyano-3,12-dioxooleana-1,9(11)-dien-28-oyl]-4(-pyridin-2-yl)-1H-imidazole (“CDDO-2P-Im”), 1-[2-Cyano-3,12-dioxooleana-1,9(11)-dien-28-oyl]-4(-pyridin-3-yl)-1H-imidazole (“CDDO-3P-Im”), or derivatives thereof.

### CROSS REFERENCE TO RELATED APPLICATIONS

This application is a continuation of U.S. patent application Ser. No. 17/594,287, filed on Oct. 8, 2021, which is a national stage entry of International Patent Application No. PCT/US2020/027588, filed on Apr. 10, 2020, which claims priority to U.S. Patent Application No. 62/832,543, filed on Apr. 11, 2019. The contents of each application are incorporated by reference herein in their entirety.

### SEQUENCE LISTING

The computer-readable Sequence Listing submitted on Mar. 24, 2026, and identified as follows: 53,422 bytes ST.26 XML document file named “Sequence_Listing.xml,” created Mar. 24, 2026, is incorporated herein by reference in its entirety.

### TECHNICAL FIELD

The present invention relates to compounds and compositions useful as irreversible inhibitors of protein targets and to methods of using such compounds and compositions in the treatment of various diseases or conditions by irreversibly inhibiting such protein targets.

### BACKGROUND

Traditional reversible inhibitors are in equilibrium with their cognate target protein(s)—continually binding, unbinding, and rebinding. On the other hand, irreversible inhibitors bind and form a permanent bond with their cognate target protein(s). As such, irreversible inhibitors may have a longer duration of action with respect to conventional inhibitors.

CDDO-Me has been shown to activate the Nrf2-Keap1 pathway in both in vitro and in vivo models through covalent binding to the key proteins. Several triterpenoids including CDDO-Me have been shown to form Michael adducts at C1 of the A ring with reactive nucleophiles, such as free cysteine thiols, on a target protein. Indeed, the proposed mechanism underlying certain effects of CDDO-Me is by the formation of these Michael adducts. This thiol-Michael reaction is intrinsically reversible and CDDO-Me has been recognized as a reversible covalent inhibitor that rapidly dissociates from its target. The protein targets and chemical mechanisms of other triterpenoids remains largely unknown.

Many diseases are associated with abnormal protein (e.g., enzyme, transcription factor, histone) activity. While reversible covalent inhibitors offer certain advantages, irreversible covalent inhibitors, which can in theory achieve complete neutralization of the target protein, may provide further advantages, including mitigation of competition by endogenous substrates for binding to target proteins and prolonged duration of action, which may provide opportunities for improved dosing regimens.

Accordingly, there remains a need to identify irreversible inhibitors of target proteins that may be useful as therapeutic agents. Identification and development of irreversible covalent inhibitors may open the door to additional or refined indications and/or improved dosing regimens.

### SUMMARY OF THE INVENTION

In one aspect, this disclosure provides protein-small molecule complex comprising a protein and a bifunctional triterpenoid covalently linked to at least one amino acid residue of the protein. In certain embodiments, this disclosure provides a Kelch-Like ECH-Associated Protein 1 (Keap1) complex comprising a Keap1 protein and a bifunctional triterpenoid covalently linked to at least one amino acid residue of the Keap1 protein. In certain other embodiments, this disclosure provides a Lon protease 1 (LONP1) complex comprising a LONP1 protein and a bifunctional triterpenoid covalently linked to at least one amino acid residue of the LONP1 protein.

In one aspect, this disclosure provides a conjugate comprising a bifunctional triterpenoid covalently linked to at least one amino acid residue of a protein. In certain embodiments, the protein is human Keap1 and the bifunctional triterpenoid is covalently linked to an amino acid residue corresponding to Cys38, Tyr85, Tyr208, Cys257, Tyr263, Cys288, Lys323, or Tyr443 of SEQ ID NO: 3. In certain embodiments, the protein is human LONP1 and the bifunctional triterpenoid is covalently linked to an amino acid residue corresponding to Lys426, Tyr473, Cys520, Cys637, Tyr673, Cys682, Lys718, or Lys896 of SEQ ID NO: 4.

In one aspect, this disclosure provides a cross-linked conjugate comprising a bifunctional triterpenoid covalently linked to at least two amino acid residues of a protein or a protein complex. In certain embodiments, the bifunctional triterpenoid is covalently linked to (i) a cysteine residue and (ii) a lysine, serine, arginine, or tyrosine residue. In some such embodiments, the protein is human Keap1 and the lysine, serine, arginine, or tyrosine residue corresponds to Tyr85, Tyr208, Tyr263, Lys323, or Tyr443 of SEQ ID NO: 3 and the cysteine residue corresponds to Cys38, Cys257, or Cys288 of SEQ ID NO: 3. In other such embodiments, the protein is human LONP1 and the lysine, serine, arginine, or tyrosine residue corresponds to Lys426, Tyr473, Tyr673, Lys718, or Lys896 of SEQ ID NO: 4 and the cysteine residue corresponds to Cys520, Cys637, or Cys682 of SEQ ID NO: 4. For example, the lysine, serine, arginine, or tyrosine residue may correspond to Tyr473 of SEQ ID NO: 4 and the cysteine residue may correspond to Cys682 of SEQ ID NO: 4

In some embodiments of any aspects disclosed herein, the bifunctional triterpenoid is 1-[2-Cyano-3,12-dioxooleana-1,9(11-dien-28-oyl) (CDDO-Im) or a pharmaceutically acceptable salt thereof; 1-[2-Cyano-3,12-dioxooleana-1,9(11)-dien-28-oyl]-4(-pyridin-2-yl)-1H-imidazole (CDDO-2P-Im) or a pharmaceutically acceptable salt thereof, 1-[2-Cyano-3,12-dioxooleana-1,9(11)-dien-28-oyl]-4(-pyridin-3-yl)-1H-imidazole (CDDO-3P-Im) or a pharmaceutically acceptable salt thereof; or analogs or derivatives of the foregoing.

In some embodiments of any aspects disclosed herein, the conjugate or cross-linked conjugate is formed in vitro. In some other embodiments of any aspects disclosed herein, the conjugate or cross-linked conjugate is formed in vivo. Thus, in some embodiments of any aspects disclosed herein, a bifunctional triterpenoid is administered to a subject, preferably orally, and a conjugate or cross-linked conjugate comprising the bifunctional triterpenoid or a derivative thereof is formed in vivo.

### BRIEF DESCRIPTION OF THE DRAWINGS

FIG. 1A depicts MS/MS spectra showing peptide ⁴⁶ASCLYGQLPK⁵⁵ from GSTP (SEQ ID NO: 1) modified by CDDO carboxylate at Cys48 with a mass addition of 491.5 amu.

FIG. 1B depicts MS/MS spectra showing cross-linking adducts formed between Ser47 and Cys48 on peptide ⁴⁶ASCLYGQLPK⁵⁵ from GSTP (SEQ ID NO: 1) with a mass addition of 473.3 amu.

FIGS. 1C and 1D show binding of CDDO-Im and CDDO-2P-Im, respectively, to GSTP at increasing drug concentrations following a 16 hour incubation at 37° C. CDDO-2P-Im most preferentially binds to Cys48 and, in comparison, to CDDO-Im, binding does not plateau above 5 μM.

FIG. 2A depicts MS/MS spectra showing an acylation of Lys195 of peptide ¹⁹¹ASSAKQR¹⁹⁷ from a human HSA protein (SEQ ID NO: 2) modified by CDDO-2P-Im. Similar adducts were also detected on Ser193, Tyr319, and Arg142.

FIGS. 3A and 3B depict representative MS/MS spectra showing that CDDO-2P-Im forms a covalent adduct with Cys288 of peptide ²⁸⁸CEILQSDSR²⁹⁶ from Keap1 (SEQ ID NO: 3) through Michael addition (3A) and an adduct with Tyr85 of peptide ⁸⁵YQDAPAAQFMAHK⁹⁷ from Keap1 (SEQ ID NO: 3) through acylation (3B).

FIGS. 4A and 4B depict representative MS/MS spectra showing that CDDO-2P-Im forms a covalent adduct with Tyr473 of peptide ⁴⁷³YSNENLDLAR⁴⁸² from LONP1 (SEQ ID NO: 4) through acylation (4A) and an adduct with Cys682 of peptide ⁶⁸⁰LCGLDESK⁶⁸⁸ from LONP1 (SEQ ID NO: 4) through Michael addition (4B).

FIG. 5A. depict representative MS/MS spectra showing that CDDO-2P-Im forms a Michael adduct with Cys52 of peptide ⁵⁰GSCFHR⁵⁵ from human peptidy-prolyl cis-trans isomerase A (PPIA_HUMAN, P62937; SEQ ID NO: 5). FIGS. 5B and 5C show the molecular function (5B) and pathway (5C) of all CDDO-2P-Im modified proteins were analyzed according to the protein class categorized by PANTHER 14.2.

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. DEFINITIONS

As used in the specification and the appended claims, unless specified to the contrary, the following terms have the meaning indicated:

The term “about” as used herein, means approximately, and in most cases within 10% of the stated value.

The term “pharmaceutically acceptable” is used adjectivally to mean that the modified noun is appropriate for use as a pharmaceutical product for human use or as a part of a pharmaceutical product for human use.

The terms “treat”, “treating” and “treatment” refer to a method of alleviating or abrogating a condition, disorder, or disease and/or the attendant symptoms thereof.

### B. METHODS OF TREATMENT

In one aspect, this disclosure provides a method for irreversibly inhibiting the activity of a protein in a patient or in a biological sample. The method comprises the step of administering to said patient or contacting said biological sample with a synthetic bifunctional triterpenoid compound, wherein the activity of the protein is irreversibly inhibited by covalently modifying a nucleophilic amino acid residue (e.g., a lysine, serine, arginine, tyrosine, histidine, glutamate, or aspartate) contained therein. In some embodiments, the nucleophilic amino acid is lysine, serine, arginine, or tyrosine. In some embodiments, the synthetic bifunctional triterpenoid compound is selected from the group consisting of: (a) 1-[2-Cyano-3,12-dioxooleana-1,9(11-dien-28-oyl) (CDDO-Im) or a pharmaceutically acceptable salt thereof; (b) 1-[2-Cyano-3,12-dioxooleana-1,9(11)-dien-28-oyl]-4(-pyridin-2-yl)-1H-imidazole (CDDO-2P-Im) or a pharmaceutically acceptable salt thereof; and (c) CDDO-AAN or a pharmaceutically acceptable salt thereof. In some preferred embodiments, the synthetic bifunctional triterpenoid compound is CDDO-2P-Im.

In some embodiments, the synthetic bifunctional triterpenoid compound is administered orally. In some embodiments, the patient is a cancer patient.

In some embodiments, the biological sample is a sample derived from a cancer patient.

In one aspect, this disclosure provides a method for treating a disease or condition mediated by a protein. The method comprises administering to a patient in need thereof a therapeutically effective amount of a synthetic bifunctional triterpenoid compound that irreversibly inhibits the protein.

In some embodiments, the synthetic bifunctional triterpenoid compound is selected from the group consisting of: 1-[2-Cyano-3,12-dioxooleana-1,9(11-dien-28-oyl) (CDDO-Im) or a pharmaceutically acceptable salt thereof and 1-[2-Cyano-3,12-dioxooleana-1,9(11)-dien-28-oyl]-4(-pyridin-2-yl)-1H-imidazole (CDDO-2P-Im) or a pharmaceutically acceptable salt thereof. In some preferred embodiments, the synthetic bifunctional triterpenoid compound is CDDO-2P-Im.

In some embodiments, the disease or condition is selected from the group consisting of cancer, cancer therapy resistance, autoimmune diseases, inflammatory diseases (e.g., Crohn's disease and other diseases associated with aberrant inflammatory responses, including neuropsychiatric disorders and, particularly, depression), neurodegenerative diseases (e.g., Alzheimer's disease (AD), Parkinson's disease (PD), amyotrophic lateral sclerosis (ALS), multiple sclerosis (MS), Huntington disease), diseases of the eye (e.g., diabetic retinopathy, macular degeneration), diseases of the lung (e.g., Chronic Obstructive Pulmonary Disease (COPD), emphysema, pulmonary fibrosis, bronchopulmonary dysplasia), diseases of the liver (e.g., chronic metabolic disease, liver injury from various toxins), atherosclerosis, chronic kidney disease (CKD), including CKD resulting from diabetes, acute renal injury, and aging. In some such embodiments, the cancer is a blood cancer, such as leukemia, lymphoma, or myeloma; in particular embodiments the blood cancer is, for example, Hodgkin or non-Hodgkin lymphoma (e.g., diffuse large B cell lymphoma or mantle cell lymphoma). In other such embodiments, the cancer involves a solid tumor, such as breast cancer, ovarian cancer, or brain cancer (e.g., medulloblastoma or glioblastoma). In some such embodiments, the cancer therapy resistance is resistance to proteasome inhibitor (PI) therapy. In some such embodiments, the cancer therapy resistance is resistance to an oral chemotherapeutic agent, such as an alkylating agent (e.g., TMZ). In some such embodiments, the cancer therapy resistance is resistance to radiation therapy.

In some embodiments, the protein is Keap1. In some embodiments, the protein is LONP1. In some embodiments, the protein is a B cell protein.

In some embodiments, the synthetic bifunctional triterpenoid compound is administered orally. In some embodiments, the patient is a cancer patient.

### C. COMPOUNDS AND COMPOSITIONS

CDDO-Im is a synthetic triterpenoid. U.S. Pat. No. 6,974,801 and WO 2004/064723, each of which are incorporated herein by reference in their entirety, describe 1-(2-cyano-3,12-dioxooleana-1,9(11)-dien-28-oyl) imidazole (CDDO-Im), which has the chemical structure:

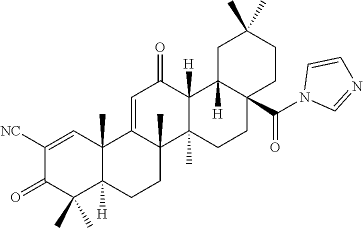

U.S. Pat. No. 9,896,475, which is incorporated herein by reference in its entirety, describes analogs and derivatives of CDDO-Im, including pyridyl analogs of CDDO-Im, which are more stable in human plasma and achieve a higher concentration in target tissues such as liver, pancreas, kidney and lungs.

Particular synthetic triterpenoids described herein include compounds having the structure of Formula A and pharmaceutically acceptable salts thereof:

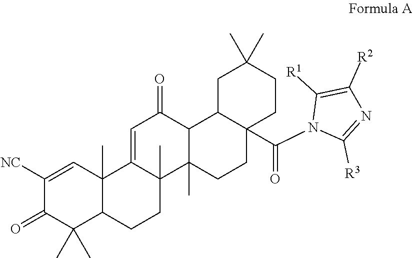

wherein one or more of R¹, R² or R³ is independently a heteroaryl group (preferably a pyridyl group), cycloalkyl group, heterocyclyl group, carboxamide group, nitrile group, haloalkyl group, or acyl group, each of which may be substituted or unsubstituted where appropriate, and the remaining R groups are hydrogen. In a particular embodiment, R² is a substituted or unsubstituted aryl group, heteroaryl group, cycloalkyl group or heterocyclyl group, and R¹ and R³ are hydrogen.

The term “heteroaryl” refers to a five- or six-membered aromatic ring structure, wherein at least one of the aromatic ring atoms is nitrogen, oxygen or sulfur, and wherein the monovalent group is composed of carbon, hydrogen, aromatic nitrogen, aromatic oxygen or aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, and chromenyl, wherein the point of attachment is one of the aromatic atoms. In particular embodiments, the heteroaryl is a pyridyl group.

“Cycloalkyl” means a non-aromatic mono- or multicyclic ring system including about 3 to about 10 carbon atoms, preferably about 5 to about 10 carbon atoms. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Heterocyclyl” or “heterocycloalkyl” means a non-aromatic saturated monocyclic or multicyclic ring system including about 3 to about 10 ring atoms, preferably about 5 to about 10 ring atoms, in which one or more of the atoms in the ring system is an element other than carbon, for example nitrogen, oxygen or sulfur, alone or in combination. Preferred heterocyclyls contain about 5 to about 6 ring atoms. The prefix aza, oxa or thia before the heterocyclyl root name means that at least a nitrogen, oxygen or sulfur atom respectively is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. Non-limiting examples of suitable bicyclic heterocyclyl rings include decahydro-isoquinoline, decahydro-[2,6]naphthyridine, and the like.

As used herein, a “carboxamide” or “carboxamide group” refers to a —C(═O)NH₂ group.

The term “nitrile” or “nitrile group” is intended to refer to a —C≡N group.

As used herein, “alkyl” or “alkyl group” includes linear or branched saturated aliphatic hydrocarbon groups having the specified number of carbon atoms. C₁₋₆ alkyl, for example, includes C₁, C₂, C₃, C₄, C₅, and C₆ alkyl groups. Examples of alkyl include, but are not limited to, methyl, ethyl, n-propyl, isopropyl, n-butyl, isobutyl, sec-butyl, t-butyl, n-pentyl, sec-pentyl, 3-(2-methyl)butyl, 2-pentyl, 2-methylbutyl, n-hexyl, and 2-methylpentyl. In particular embodiments, an alkyl of this invention is a C₁₋₆ alkyl, C₁₋₅ alkyl, C₁₋₄ alkyl, C₁₋₃ alkyl, or C₁₋₂ alkyl.

The term “haloalkyl group” refers to a linear or branched alkyl group substituted by one or more halogen atoms, the same or different, optionally selected from fluorine, chlorine, bromine, and iodine. Examples of this group include fluoromethyl, difluoromethyl, trifluoromethyl, trichloromethyl, 2,2,2-trifluoroethyl, 2,2,2-trichloroethyl, 2,2,3,3-tetrafluoropropyl, 2,2,3,3,3-pentafluoropropyl.

“Acyl,” as used herein alone or as part of another group, refers to a —C(═O)R radical, where R is, e.g., an aryl, alkyl, alkenyl, alkynyl, cycloalkyl, or haloalkyl group. When the R group contains a halogen, the group is termed a “haloacyl” group. An example is a trifluoroacetyl group.

The term “aryl” refers to a monovalent group with an aromatic carbon atom as the point of attachment, said carbon atom forming part of a five- or six-membered aromatic ring structure wherein the ring atoms are all carbon, and wherein the monovalent group is composed of carbon and hydrogen. Non-limiting examples of aryl groups include phenyl, methylphenyl, (dimethyl)phenyl, -ethylphenyl, propylphenyl, —C₆H₄CH(CH₃)₂, —C₆H₄CH(CH₂)₂, methylethylphenyl, vinylphenyl, naphthyl, and the monovalent group derived from biphenyl. In particular embodiments, the aryl is a phenyl group.

As used herein, “alkenyl” or “alkenyl group” refers to an unsaturated branched, straight-chain or cyclic monovalent hydrocarbon radical having at least one carbon-carbon double bond derived by the removal of one hydrogen atom from a single carbon atom of a parent alkene. The radical may be in either the cis or trans conformation about the double bond(s). Examples of alkenyl include, but are not limited to, ethenyl, propenyls, such as prop-1-en-1-yl, prop-1-en-2-yl, prop-2-en-1-yl, prop-2en-2-yl, cycloprop-1-en-1-yl; cycloprop-2-en-1-yl; butenyls such as but-1-en-1-yl, but-1-en-2-yl, 2-methyl-prop-1-en-lyl, but-2-en-1-yl, but-2-en-1-yl, but-2-en-2-yl, buta-1,3-dien1-yl, beta-1,3-dien-2-yl, cyclobut-1-en-1-yl, cyclobut-1-en3-yl, cyclobuta-1,3-dien-1-yl.

As used herein, an “alkynyl” or “alkynyl group” refers to an unsaturated branched, straight-chain or cyclic monovalent hydrocarbon radical having at least one carbon-carbon triple bond derived by the removal of one hydrogen atom from a single carbon atom of a parent alkyne. Examples of alkynyl groups include, but are not limited to, ethynyl, propynyl, butynyls, propargyl, and the like.

Any of the groups described herein may be unsubstituted or optionally substituted. When modifying a particular group, “substituted” means that the group the term modifies may, but does not have to, be substituted. Substitutions include the replacement of an available hydrogen with an alkyl, alkenyl, alkynyl, aryl, haloalkyl, haloacyl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, acyl, halo, nitro, cyano, cyanoalkyl, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, or heterocyclyl.

Any undefined valency on an atom of a structure shown in this application implicitly represents a hydrogen atom bonded to the atom.

In some embodiments of any aspects disclosed herein, a synthetic triterpenoid has the structure of Formula B and pharmaceutically acceptable salts thereof:

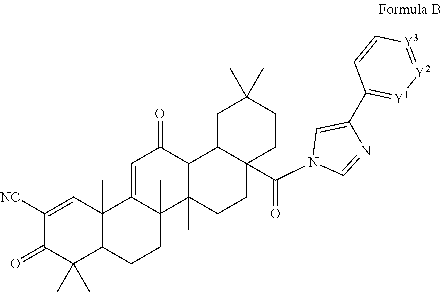

wherein one of Y¹, Y², or Y³, is N and the remaining Y groups are each CH. In a particular embodiment, Y¹ is N and Y² and Y³ are CH. In another particular embodiment, Y² is N and Y¹ and Y³ are CH. In another particular embodiment, Y³ is N and Y¹ and Y² are CH.

A particularly preferred synthetic bifunctional triterpenoid compound is 1-[2-Cyano-3,12-dioxooleana-1,9(11)-dien-28-oyl]-4(-pyridin-2-yl)-1H-imidazole (CDDO-2P-Im), which is depicted structurally as:

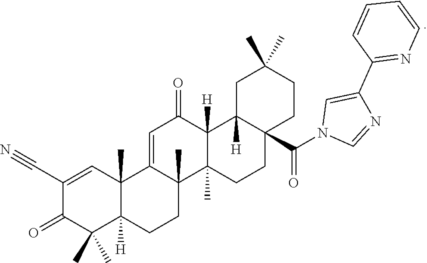

Another particularly preferred synthetic bifunctional triterpenoid is 1-[2-Cyano-3,12-dioxooleana-1,9(11)-dien-28-oyl]-4(-pyridin-3-yl)-1H-imidazole (CDDO-3P-Im), which is depicted structurally as:

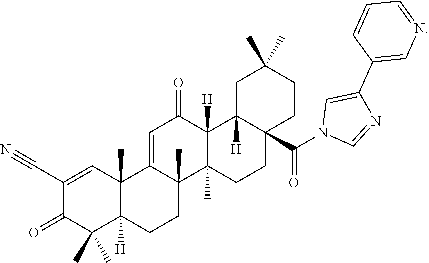

Another bifunctional triterpenoid can be prepared by reacting CDDO acid chloride with NH₂CH₂CN (aminoacetonitrile) in the presence of a base. The product of this reaction is referred to herein as “CDDO-AAN.”

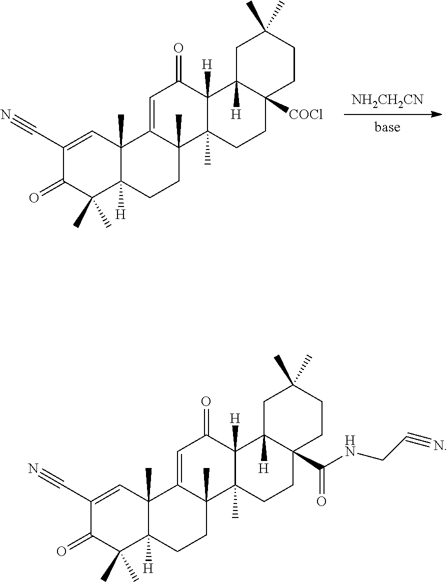

In some embodiments of any aspects disclosed herein, a synthetic bifunctional triterpenoid compound may be present in a pharmaceutical composition in the form of acid or base addition salts. Acid addition salts may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Suitable base addition salts include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” is intended to encompass any and all acceptable salt forms.

Pharmaceutical compositions disclosed herein comprise a synthetic bifunctional triterpenoid compound, preferably CDDO-2P-Im or a pharmaceutically acceptable salt thereof. In some embodiments, the pharmaceutical composition is an oral dosage form, preferably a solid oral dosage form (e.g., a tablet). In some such embodiments, the solid oral dosage form may comprise pharmaceutically acceptable excipients such as excipients that function as binders, glidants, lubricants, and fillers. Thus, a solid oral dosage form comprising a synthetic bifunctional triterpenoid compound, further optionally comprises one or more conventional pharmaceutically acceptable excipients.

### D. CONJUGATES

In one aspect, this disclosure provides a conjugate comprising a protein having a nucleophilic residue, wherein the nucleophilic residue is covalently, and irreversibly, bonded to a synthetic bifunctional triterpenoid. Exemplary nucleophilic residues include lysine, serine, arginine, tyrosine, histidine, glutamate, and aspartate. Exemplary bifunctional triterpenoids include CDDO-Im, CDDO-2P-Im, CDDO-AAN, and derivatives thereof.

In one aspect, this disclosure provides a conjugate comprising a protein having a lysine, serine, arginine, or tyrosine residue, wherein the lysine, serine, arginine, or tyrosine residue is covalently, and irreversibly, bonded to a synthetic triterpenoid; wherein said conjugate has a structure corresponding to Formula (I):

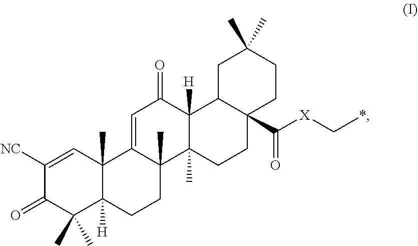

wherein * represents the point of attachment through the lysine, serine, arginine, or tyrosine residue and X represents O or N(H).

In some embodiments, X is O. In some embodiments, X is N(H).

In some embodiments, the residue bonded to the synthetic triterpenoid is a lysine residue. In some embodiments, the residue bonded to the synthetic triterpenoid is a serine residue. In some embodiments, the residue bonded to the synthetic triterpenoid is an arginine residue. In some embodiments, the residue bonded to the synthetic triterpenoid is a tyrosine residue.

Depending upon the nature of the leaving group of the bifunctional triterpenoid, * may comprise a direct bond to the amino acid residue or, alternatively a linker, such as a one, two, or three atom linker between the triterpenoid skeleton and the amino acid residue. In some such embodiments, * comprises —C(═NH)—.

In one aspect, this disclosure provides a cross-linked conjugate comprising one or more proteins, said one or more proteins having (i) a cysteine residue and (ii) a lysine, serine, arginine, or tyrosine residue, wherein the lysine, serine, arginine, or tyrosine residue is covalently, and irreversibly, bonded to a synthetic, bifunctional triterpenoid; wherein said conjugate has a structure corresponding to Formula (II):

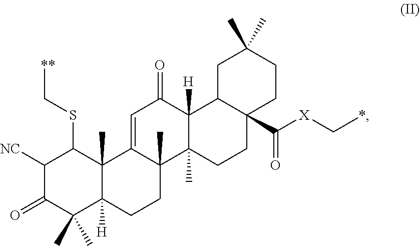

wherein * represents the point of attachment through the lysine, serine, arginine, or tyrosine residue, ** represents the point of attachment through the cysteine residue, and X represents O or N(H).

In some embodiments, the cross-linked conjugate comprises a single protein. In some embodiments, the cross-linked conjugate comprises two proteins.

In some embodiments, the cysteine residue is in the same protein as the lysine, serine, arginine, or tyrosine residue. In some such embodiments, the cysteine residue is adjacent to the lysine, serine, arginine, or tyrosine residue. In some embodiments, the cysteine residue is in a different protein from the lysine, serine, arginine, or tyrosine residue.

In some embodiments, X is O. In some embodiments, X is N(H).

In some embodiments, the residue bonded to the synthetic, bifunctional triterpenoid is a lysine residue. In some embodiments, the residue bonded to the synthetic, bifunctional triterpenoid is a serine residue. In some embodiments, the residue bonded to the synthetic, bifunctional triterpenoid is an arginine residue. In some embodiments, the residue bonded to the synthetic, bifunctional triterpenoid is a tyrosine residue.

Depending upon the nature of the leaving group of the bifunctional triterpenoid, * may comprise a direct bond to the amino acid residue or, alternatively a linker, such as a one, two, or three atom linker between the triterpenoid skeleton and the amino acid residue. In some such embodiments, * comprises —C(═NH)—.

Data presented herein show that multiple covalent adducts were detected by mass spectrometric analysis when the bifunctional triterpenoids were incubated with various proteins in vitro. Without wishing to be bound by any particular theory, it is believed that a simple Michael adduct is initially formed through conjugation of cysteine residues to the α,β unsaturated ketone located in the A ring. Further hydrolysis of the imidazole ester resulted in the CDDO carboxylic acid adduct. Due to the reversible nature of the thiol addition, only low levels of this adduct were detected. Additionally, acylation of other amino acid residues (lysine, serine, tyrosine, and arginine) could occur at the C28 carbonyl carbon of bifunctional triterpenoids. Finally, cross-linking adducts could be formed by conjugation to cysteine residues at C1 and acylation to other amino acid residues at C28. The cross-linking could either occur on the same peptide containing two adjacent reactive amino acid residues or two different peptides. The latter could potentially result in cross-linking two different proteins.

An exemplary scheme is depicted below as Scheme 1.

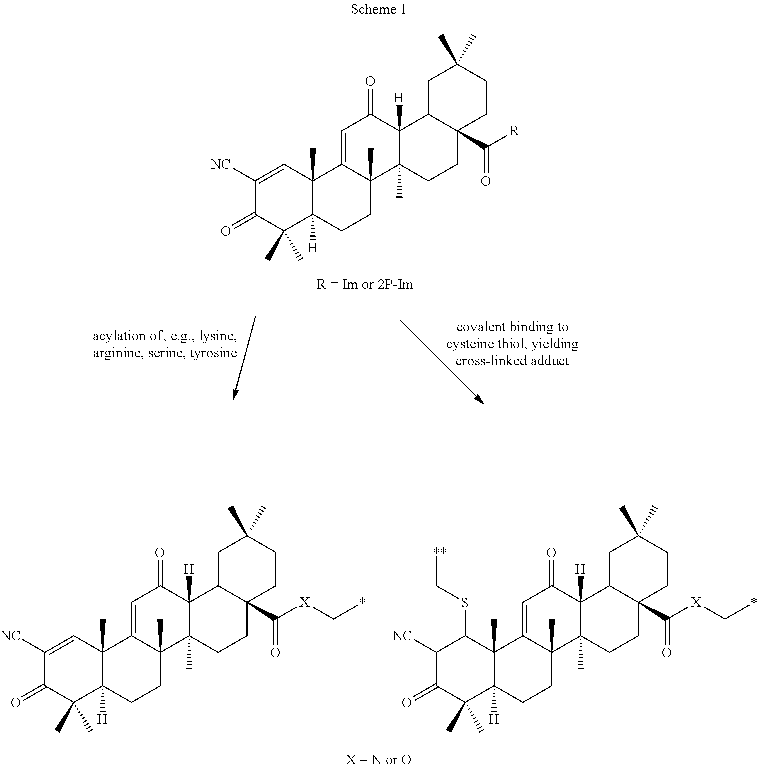

In one aspect, this disclosure provides a conjugate comprising a bifunctional triterpenoid covalently linked to at least one amino acid residue of a protein.

In one aspect, this disclosure provides a cross-linked conjugate comprising a bifunctional triterpenoid covalently linked to at least two amino acid residues of a protein or protein complex.

The following Table illustrates exemplary protein sequences modified by bifunctional triterpenoids, such as CDDO-Im, CDDO-2P-Im, and CDDO-3P-Im.

| Name | Sequence | SEQ ID NO: |
|---|---|---|
| GSTP1 | MPPYTVVYFPVRGRCAALRMLLADQGQSWKEEVVTVETWQESLKASC | 1 |
| (human) | LYGQLPKFQDGDLTLYQSNTILRHLGRTLGLYGKDQQEAALDMVNDG | |
| (P09211) | VEDLRCKYISLIYTNYEAGKDDYVKALPGQLKPFETLLSQNQGGKTF | |
| | IVGDQISFADYNLLDLLLIHEVLAPGCLDAFPLLSAYVGRLSARPKL | |
| | KAFLASPEYVNLPINGNGKQ | |
| serum | DAHKSEVAHRFKDLGEENFKALVLIAFAQYLQQCPFEDHVKLVNEVT | 2 |
| albumin | EFAKTCVADESAENCDKSLHTLFGDKLCTVATLRETYGEMADCCAKQ | |
| (human) | EPERNECFLQHKDDNPNLPRLVRPEVDVMCTAFHDNEETFLKKYLYE | |
| (P02768)^{a} | IARRHPYFYAPELLFFAKRYKAAFTECCQAADKAACLLPKLDELRDE | |
| | GKASSAKQRLKCASLQKFGERAFKAWAVARLSQRFPKAEFAEVSKLV | |
| | TDLTKVHTECCHGDLLECADDRADLAKYICENQDSISSKLKECCEKP | |
| | LLEKSHCIAEVENDEMPADLPSLAADFVESKDVCKNYAEAKDVFLGM | |
| | FLYEYARRHPDYSVVLLLRLAKTYETTLEKCCAAADPHECYAKVFDE | |
| | FKPLVEEPQNLIKQNCELFEQLGEYKFQNALLVRYTKKVPQVSTPTL | |
| | VEVSRNLGKVGSKCCKHPEAKRMPCAEDYLSVVLNQLCVLHEKTPVS | |
| | DRVTKCCTESLVNRRPCFSALEVDETYVPKEFNAETFTFHADICTLS | |
| | EKERQIKKQTALVELVKHKPKATKEQLKAVMDDFAAFVEKCCKADDK | |
| | ETCFAEEGKKLVAASQAALGL | |
| KEAP1 | MQPDPRPSGAGACCRFLPLQSQCPEGAGDAVMYASTECKAEVTPSQH | 3 |
| (human) | GNRTFSYTLEDHTKQAFGIMNELRLSQQLCDVTLQVKYQDAPAAQFM | |
| (Q14145) | AHKVVLASSSPVFKAMFTNGLREQGMEVVSIEGIHPKVMERLIEFAY | |
| | TASISMGEKCVLHVMNGAVMYQIDSVVRACSDFLVQQLDPSNAIGIA | |
| | NFAEQIGCVELHQRAREYIYMHFGEVAKQEEFFNLSHCQLVTLISRD | |
| | DLNVRCESEVFHACINWVKYDCEQRRFYVQALLRAVRCHSLTPNFLQ | |
| | MQLQKCEILQSDSRCKDYLVKIFEELTLHKPTQVMPCRAPKVGRLIY | |
| | TAGGYFRQSLSYLEAYNPSDGTWLRLADLQVPRSGLAGCVVGGLLYA | |
| | VGGRNNSPDGNTDSSALDCYNPMTNQWSPCAPMSVPRNRIGVGVIDG | |
| | HIYAVGGSHGCIHHNSVERYEPERDEWHLVAPMLTRRIGVGVAVLNR | |
| | LLYAVGGFDGTNRLNSAECYYPERNEWRMITAMNTIRSGAGVCVLHN | |
| | CIYAAGGYDGQDQLNSVERYDVETETWTFVAPMKHRRSALGITVHQG | |
| | RIYVLGGYDGHTFLDSVECYDPDTDTWSEVTRMTSGRSGVGVAVTME | |
| | PCRKQIDQQNCTC | |
| LONP1 | MAASTGYVRLWGAARCWVLRRPMLAAAGGRVPTAAGAWLLRGQRTCD | 4 |
| (human) | ASPPWALWGRGPAIGGQWRGFWEASSRGGGAFSGGEDASEGGAEEGA | |
| (P36776) | GGAGGSAGAGEGPVITALTPMTIPDVFPHLPLIAITRNPVFPRFIKI | |
| | IEVKNKKLVELLRRKVRLAQPYVGVFLKRDDSNESDVVESLDEIYHT | |
| | GTFAQIHEMQDLGDKLRMIVMGHRRVHISRQLEVEPEEPEAENKHKP | |
| | RRKSKRGKKEAEDELSARHPAELAMEPTPELPAEVLMVEVENVVHED | |
| | FQVTEEVKALTAEIVKTIRDIIALNPLYRESVLQMMQAGQRVVDNPI | |
| | YLSDMGAALTGAESHELQDVLEETNIPKRLYKALSLLKKEFELSKLQ | |
| | QRLGREVEEKIKQTHRKYLLQEQLKIIKKELGLEKDDKDAIEEKFRE | |
| | RLKELVVPKHVMDVVDEELSKLGLLDNHSSEFNVTRNYLDWLTSIPW | |
| | GKYSNENLDLARAQAVLEEDHYGMEDVKKRILEFIAVSQLRGSTQGK | |
| | ILCFYGPPGVGKTSIARSIARALNREYFRFSVGGMTDVAEIKGHRRT | |
| | YVGAMPGKIIQCLKKTKTENPLILIDEVDKIGRGYQGDPSSALLELL | |
| | DPEQNANFLDHYLDVPVDLSKVLFICTANVTDTIPEPLRDRMEMINV | |
| | SGYVAQEKLAIAERYLVPQARALCGLDESKAKLSSDVLTLLIKQYCR | |
| | ESGVRNLQKQVEKVLRKSAYKIVSGEAESVEVTPENLQDFVGKPVFT | |
| | VERMYDVTPPGVVMGLAWTAMGGSTLFVETSLRRPQDKDAKGDKDGS | |
| | LEVTGQLGEVMKESARIAYTFARAFLMQHAPANDYLVTSHIHLHVPE | |
| | GATPKDGPSAGCTIVTALLSLAMGRPVRQNLAMTGEVSLTGKILPVG | |
| | GIKEKTIAAKRAGVTCIVLPAENKKDFYDLAAFITEGLEVHFVEHYR | |
| | EIFDIAFPDEQAEALAVER | |
| PPIA | MVNPTVFFDIAVDGEPLGRVSFELFADKVPKTAENFRALSTGEKGFG | 5 |
| (human) | YKGSCFHRIIPGFMCQGGDFTRHNGTGGKSIYGEKFEDENFILKHTG | |
| (P62937) | PGILSMANAGPNTNGSQFFICTAKTEWLDGKHVVFGKVKEGMNIVEA | |
| | MERFGSRNGKTSKKITIADCGQLE | |

^{a}The first 24 amino acid residues in P02768 comprise a signalling peptide. The HSA protein depicted in SEQ ID NO: 2 contains 585 amino acids (corresponding to 25-609 of P02768).

In certain embodiments, the protein is human Keap1 and the bifunctional triterpenoid is covalently linked to an amino acid residue corresponding to Cys38, Tyr85, Tyr208, Cys257, Tyr263, Cys288, Lys323, or Tyr443 of SEQ ID NO: 3. In some such embodiments, the bifunctional triterpenoid forms a covalent adduct with Cys288 through Michael addition and an adduct with Tyr85 through acylation.

In certain embodiments, the protein is human LONP1 and the bifunctional triterpenoid is covalently linked to an amino acid residue corresponding to Lys426, Tyr473, Cys520, Cys637, Tyr673, Cys682, Lys718, or Lys896 of SEQ ID NO: 4. In some such embodiments, the bifunctional triterpenoid forms a covalent adduct with Cys682 through Michael addition and an adduct with Tyr473 through acylation.

In certain embodiments, the protein is human PPIA and the bifunctional triterpenoid is covalently linked to an amino acid residue corresponding to Cys52, Cys62, Cys115, or Cys161 of SEQ ID NO: 5. In some such embodiments, the bifunctional triterpenoid forms a covalent adduct with Cys52 through Michael addition.

### E. EXAMPLES

The protein targets and chemical mechanisms of bifunctional triterpenoids, such as CDDO-Im, have not been heretofore elucidated. Data presented herein show that bifunctional triterpenoids form multiple adducts with a variety of amino acids on model proteins such as HSA and GSTP. Further studies were carried out to identify intracellular protein targets and characterize the structure of the covalent adducts formed by a range of bifunctional triterpenoids. Data presented herein show that bifunctional triterpenoids such as CDDO-Im and CDDO-2P-Im can form (i) a simple Michael adduct with cysteine, (ii) a cross-linking adduct (e.g., between adjacent cysteine and serine/arginine/lysine residues), and (iii) an acylation adduct formed with arginine residues.

### Materials and General Methods

Triterpenoids used in the following examples included: CDDO-Me (1), CDDO-Im (2), CDDO-2P-Im (3), and CDDO-3P-Im (4).

Chemicals. Bifunctional Triterpenoids (2-4) were prepared as previously described. CDDO-Me and HSA (97-99% pure) was purchased from Sigma-Aldrich, trypsin from Promega (Madison, WI), liquid chromatography-mass spectrometry (LC-MS) grade solvents from Fisher Scientific UK Ltd (Loughborough, Leicestershire), and all other standard reagents from Sigma-Aldrich.

Modification of His-GSTP by triterpenoids. His-GSTP was expressed in E. coli as described previously. Purified His-GSTP captured on nickel beads was incubated with a range of concentrations of CDDO-Im (50 nM-10 μM) in phosphate buffer, pH 7.4 for 16 h. CDDO-Me (0.5 mM) was used as a positive control. The beads were then washed 5 times with 1000 μL phosphate buffer. The protein was subjected to on-bead tryptic digestion. In brief, a suspension beads in 30 μL of 50 mM ammonium bicarbonate buffer was incubated with 20 ng of trypsin for 16 h at 37° C. and the digest was analyzed by LC-MS/MS.

Modification of HSA by triterpenoids. Triterpenoids were dissolved in DMSO to make up 1 mM stock solutions, followed by dilutions in phosphate buffer (10 mM, pH 7.4) and incubated with recombinant HSA (0.6 mM, 50 μL) in sealed Eppendorf tubes at 37° C. for 16 h. The molar ratios of drug to protein were 0.00001:1, 0.0001:1, 0.001:1, 0.01:1, 0.1:1, and 1:1. Protein was precipitated twice with 9 volumes of ice-cold methanol to remove free drug, resuspended in 50 μL phosphate buffer and then reduced with 10 mM dithiothreitol (15 min) and alkylated with 55 mM iodoacetamide (15 min) at room temperature. The protein was precipitated with methanol once more and finally dissolved in 100 μL 50 mM ammonium hydrogencarbonate, and 165 μg (1.25 nmol) of protein was digested with 1.6 μg trypsin overnight at 37° C.

To examine the time-dependent modification of HSA by triterpenoids, 10 μM triterpenoids was incubated with HSA (0.6 mM, 300 μL) at 37° C. Aliquots of 50 μL were removed after 10, 30, 60, and 180 minutes and processed for LC-MS/MS analysis.

Modification of Keap1 by triterpenoids. Triterpenoids were dissolved in DMSO to make up 1 mM stock solutions, followed by dilutions in phosphate buffer (10 mM, pH 7.4) and incubated with recombinant Keap1 proteins produced in house at 37° C. for 16 h. The final concentration of triterpenoids was 100 μM. The mixture was then purified by 1D-gel electrophoresis using an established protocol, followed by in gel-digestion. The digests were further purified by C18-ziptiping and analyzed by LC-MS/MS.

Modification of LONP by triterpenoids. The LONP protein (Professor John Letterio) was first purified by buffer exchange using spin filters (3K MWCO). 10 μg protein was then incubated with CDDO-2P-Im (100 μM) in phosphate buffer (10 mM, pH 7.4) at 37° C. for 16 h. The mixture was then purified by 1D-gel electrophoresis using an established protocol, followed by in gel-digestion. The digests were further purified by C18-ziptiping and analyzed by LC-MS/MS.

Modification of B cell intracellular proteins by triterpenoids. Epstein-Barr virus-transformed B-cell lines were generated with previously described methods by incubating PBMC with supernatant from the Epstein-Barr virus-producing cell line B9-58. B cells were maintained in F1 medium at 37° C., 5% CO₂. Cells were plated into an 8-well plate at a concentration of 1×10⁶ cells/ml and treated with triterpenoids at different concentrations (0, 1, 10 μM) for 16 hours. 200 μL cells were then plated (1×10⁵ cells/well) into a 96-well plate, [³H]-thymidine (0.5 μCi/well) was then added and incubated for 16 hours. Cell was harvested and the proliferative responses were measured by [³H]-thymidine incorporation. For the remaining cells, supernatants were removed for ELISA (TNFα, IL10, and IFNγ) and proteomics analysis. The cells were then washed in PBS and lysed with 2D lysis buffer (40 mM Tris base, 7M urea, 2M thiourea, 4% (w/v) CHAPS, and 1% DTT). Cell lysates were sonicated and centrifuged at 18000 rpm for 20 minutes at ° C.4. Supernatants were collected and the protein concentrations were measured by BCA assay. Proteins were denatured and processed for mass spectrometric analysis.

LC-MS/MS analysis of triterpenoids protein adducts. The tryptic peptides were analyzed by a Triple TOF 5600 mass spectrometer (Sciex). Samples were reconstituted in 50 μL 0.1% formic acid (FA) and 2 μL of samples were delivered into the instrument using an Eksigent Nano-LC system mounted with a nanoACQUITY UPLC Symmetry C18 Trap Column and an analytical BEH C18 nanoACQUITY Column (Waters, MA, USA). A NanoSpray III source was fitted with a 10 m inner diameter PicoTip emitter (New Objective). Samples were loaded in 0.1% formic acid onto the trap, which was then washed with 2% ACN/0.1% FA for 10 min at 2 L/min before switching in-line with the analytical column. A gradient of 2-50% (v/v) ACN/0.1% (v/v) FA over 90 min was applied to the column at a flow rate of 300 nL/min. Spectra were acquired automatically in positive ion mode using information-dependent acquisition, using mass ranges of 400-1600 amu in MS and 100-1400 amu in MS/MS. Up to 25 MS/MS spectra were acquired per cycle (approximately 10 Hz) using a threshold of 100 counts per s, with dynamic exclusion for 12 s and rolling collision energy. Sequence coverage was determined using ProteinPilot software, v4.0 and the most recent version of the SwissProt database.

### Example 1. Triterpenoid Modified-GSTP

Mass spectrometric analysis of tryptic digests in the absence of triterpenoids identified the unmodified Cys48-containing peptide 46-55 of SEQ ID NO: 1 (⁴⁶ASCLYGQLPK⁵⁵). The peptide corresponding to a doubly charged ion of m/z 568.9 was detected, and the peptide sequence was confirmed by a series of y product ions (m/z 244.2, 357.3, 542.4, 705.5, 818.7, 978.7, and 1065.8) on the MS/MS spectra (data not shown).

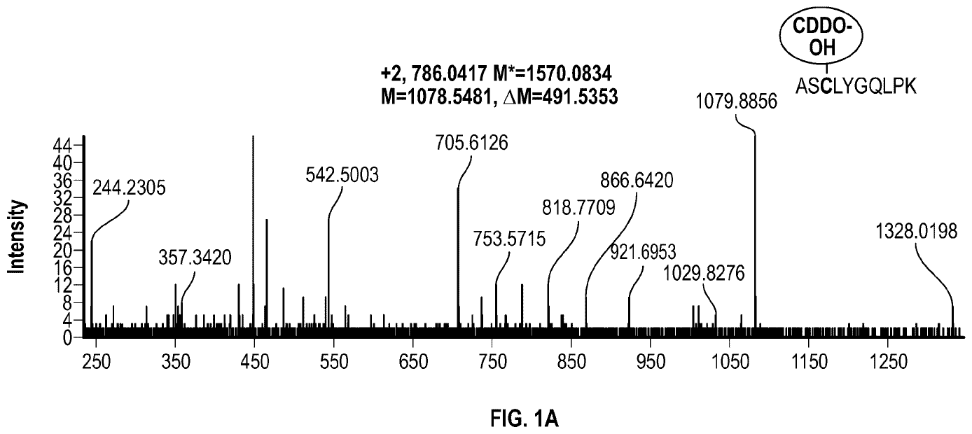

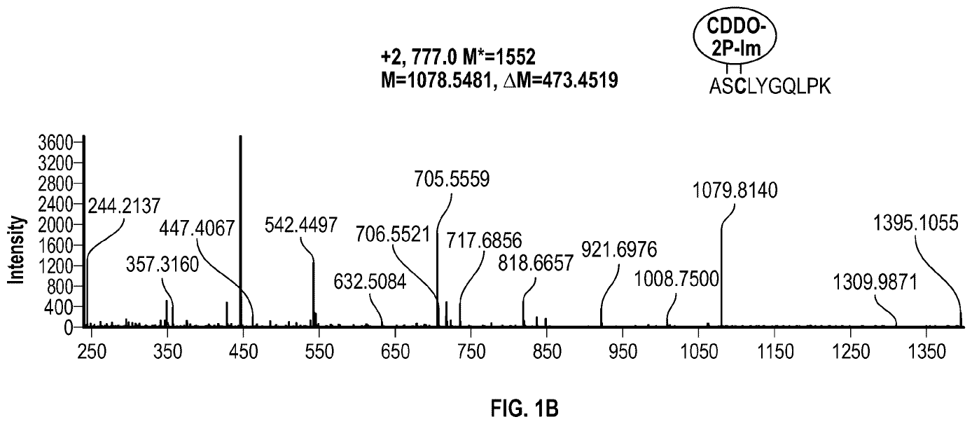

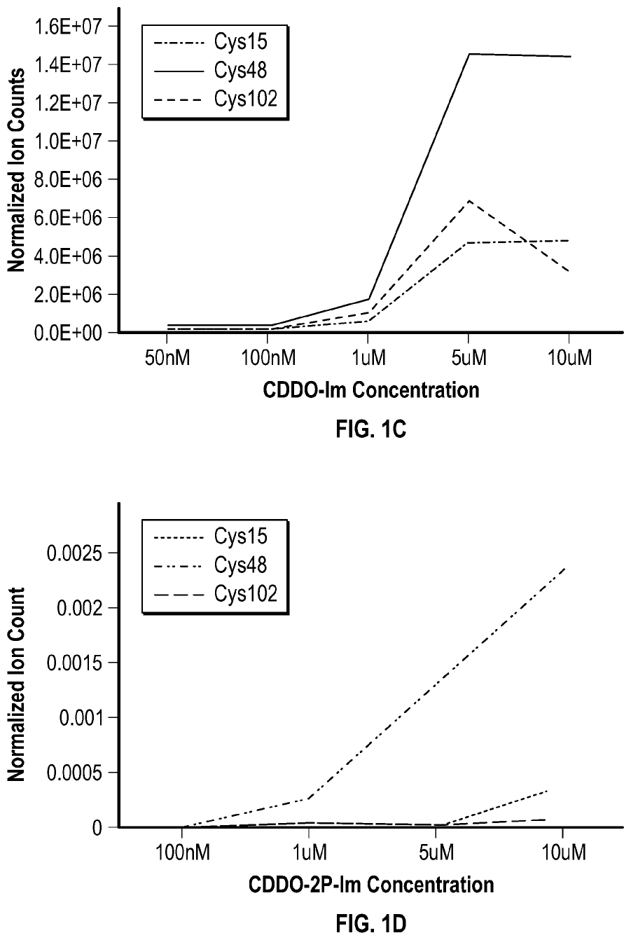

LC-MS/MS analysis of the tryptic digests of CDDO-Im treated GSTP revealed multiple types of adducts, including a simple Michael adduct formed with cysteine, a cross-linking adduct, and an acylation adduct formed with arginine residues (Table 1). A simple Michael addition of cysteine to CDDO-Im followed by the hydrolysis of the imidazole ester resulted in an adduct with a mass addition of 491.5 amu. FIG. 1A shows a representative MS/MS spectrum for a doubly charged ion at m/z 786.04, corresponding to the tryptic peptide corresponding to residues 46-55 of SEQ ID NO: 1 (⁴⁶ASCLYGQLPK⁵⁵) with an additional mass of 491.5 amu. The peptide sequence was confirmed by partial singly charged y and b series ions. The modification site was confirmed by the presence of b3* (m/z 753.57), b4* (m/z 866.64), b5* (m/z 1029.8276), and b8*(m/z 1328.0198), all with adduction of 491.5 amu. The presence of a fragment ion of m/z 464.4492 and 447.4189 that derived from CDDO carboxylic acid provided further evidence of the modification. This adduct was only detected on Cys48, no other sites were identified.

Cross-linking adducts are anticipated due to the presence of the bifunctional groups on CDDO-Im. An abundant doubly charged ion at m/z 777.0 was detected, corresponding to the tryptic peptide corresponding to residues 46-55 of SEQ ID NO: 1 (⁴⁶ASCLYGQLPK⁵⁵) with an additional mass of 473.5 amu. The cross-linking adduct could be formed by reaction of CDDO-Im with Cys48 and the adjacent serine residue in the sequence ⁴⁶ASCLYGQLPK⁵⁵ (residues 46-55 of SEQ ID NO: 1). A typical MS/MS spectrum representing the tryptic peptide ⁴⁶ASCLYGQLPK⁵⁵ (residues 46-55 of SEQ ID NO: 1) with a mass addition of 473.5 amu is shown in FIG. 1B. The peptide sequence was confirmed by a series of y product ions, and the cross-linking was evidenced by the presence of b2*(m/z 632.5084, Ser47) and y8* (m/z 1395.1055, Cys48), both with mass increment of 473.5 amu. The cross-linking adducts were also detected on multiple sites including Arg14-Cys15 and Arg101-Cys102 (Table 1). Similar to the modifications observed with Ser47, a stable adduct derived from an arginine residue was also detected (Arg187, ¹⁸⁴LSARPK¹⁸⁹) when high concentration of CDDO-Im was used (500 μM).

TABLE 1
CDDO-Im modified GSTP peptides detected in vitro^{a}

| Amino | | | | |
|---|---|---|---|---|
| acid | Peptide sequence of SEQ ID NO: 1^{b} | | m/z | RT |
| Cys15 | 13-19 | GR*C*AALR | 407.2529 | 43.04 |
| Cys15 | 15-19 | C*AALR | 503.8094 | 44.87 |
| Cys48 | 46-55 | AS*C*LYGQLPK | 776.958 | 46.82 |
| Cys102 | 83-103 | DQQEAALDMVNDGVEDLR*C*K | 706.238 | 47.398 |
| Arg187 | 184-189 | LSAR*PK | 572.9 | 45.57 |

^{a}CDDO-Im (50 nM-10 μM) was incubated with GSTP in phosphate buffer, pH 7.4 for 16 h.
^{b}The modified amino acid was labelled with *.

Covalent binding of CDDO-Im to GSTP was concentration-dependent with adducts being detectable at the lowest concentration of CDDO-Im (50 nM). See FIG. 1C. The crosslinking adducts appeared to be the major adducts formed between CDDO-Im and GSTP, likely due to the adduct stabilization through acylation at C28.

CDDO-2P-Im was found to bind to GSTP in a similar manner. See FIG. 1D. These data demonstrate that CDDO-2P-Im has potential to not only form simple Michael adducts through conjugation of cysteine residues to the α,β unsaturated ketone located in the A ring, but also to for cross-linking adducts (e.g., via acylation of nucleophilic residues on the same or different peptides).

### Example 2. Triterpenoid Modified-HSA

LC-MS/MS analysis of the tryptic digests revealed 23 CDDO-modified peptides, including peptides containing lysine (9), arginine (2), serine (7) or tyrosine (5) residues after incubation at the highest concentration of CDDO-2P-Im (500 μM) (Table 2).

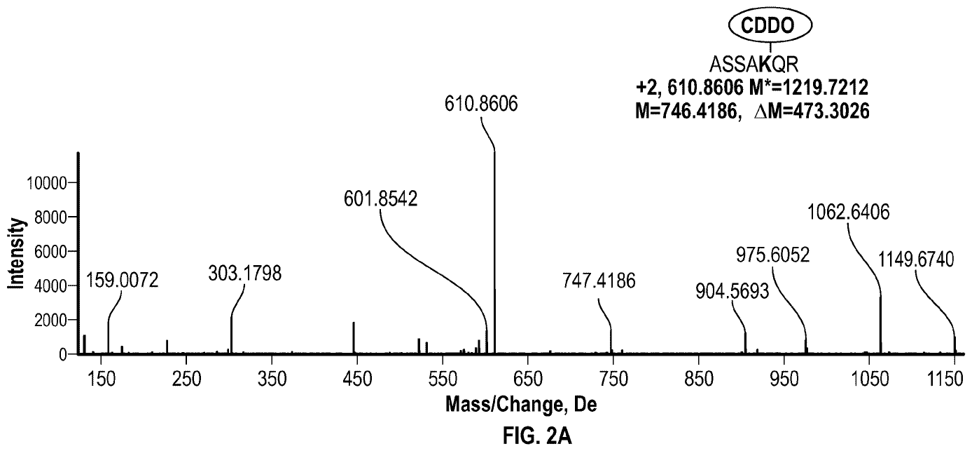

A typical MS/MS spectrum representing the tryptic peptide corresponding to 191-197 of SEQ ID NO: 2 (191ASSAK*QR¹⁹⁷) with a mass addition of 473.3 amu is shown in FIG. 2A. A missed cleavage at the proposed site of covalent binding and the presence of y3* (m/z 904.57), y4* (m/z 975.57), y5* (m/z 1062.64), and y6* (m/z 1149.67) ions provided firm evidence of acylation at Lys195. Similar to the modifications observed with lysine, stable adducts derived from serine, tyrosine, and arginine were also detected on multiple sites (Table 2).

The modification of HSA by CDDO-Im was concentration- and time-dependent. A semi-quantitative analysis of modification at each site was performed by determining the area under the curve for the extracted masses of the modified peptides, followed by normalization of the ion intensity using the total ion count for the sample. A concentration- and time-dependent increase in normalized ion counts was observed for each modified peptide.

TABLE 2
Triterpenoid modified HSA peptides detected in vitro^{a}

| Amino acid | Peptide sequence of SEQ ID NO: 2^{b} | | m/z | RT |
|---|---|---|---|---|
| K137 | 137-144 | K*YLYEIAR | 764.9434 | 82.06 |
| K195 | 191-197 | ASSAK*QR | 610.8541 | 68.57 |
| K199 | 198-205 | LK*CASLQK | 710.9154 | 74.6 |
| K413 | 411-414 | YTK*K | 506.8073 | 72.22 |
| K432 | 429-436 | NLGK*VGSK | 638.3876 | 77.25 |
| K524 | 522-525 | QIK*K | 495.3224 | 72.87 |
| K525 | 525-534 | K*QTALVELVK | 801.4988 | 82.61 |
| K536 | 535-538 | HK*PK | 491.8 | 66.39 |
| K541 | 539-545 | ATK*EQLK | 645.8875 | 73.81 |
| R186 | 182-190 | LDELR*DEGK | 516.6239 | 67.25 |
| R222 | 219-225 | LSQR*FPK | 674.75 | 80.01 |
| S193 | 191-195 | ASS*AK | 468.7725 | 81.83 |
| S202 | 200-205 | CAS*LQK | 561.8137 | 75.9 |
| S220 | 219-222 | LS*QR | 488.796 | 72.97 |
| S232 | 226-233 | AEFAEVS*K | 677.3669 | 79.01 |
| S435 | 433-436 | VGS*K | 432.2647 | 73.52 |
| S470 | 467-472 | TPVS*DR | 574.3217 | 71.09 |
| S489 | 485-500 | RPCFS*ALEVDETYVPK | 776.4013 | 81 |
| Y140 | 138-145 | YLY*EIAR | 700.8973 | 94.19 |
| Y161 | 160-162 | RY*K | 470.2868 | 76.7 |
| Y319 | 318-323 | NY*AEAK | 584.815 | 83.05 |
| Y353 | 352-359 | TY*ETTLEK | 729.3921 | 85.8 |
| Y411 | 411-413 | Y*TK | 442.2242 | 72.97 |

^{a}Triterpenoids (500 μM CDDO-Im, CDDO-2P-Im, or CDDO-3P-Im) were incubated with HSA in phosphate buffer, pH 7.4 for 16 h.
^{b}The modified amino acid was labelled with *.

### Example 3. Triterpenoid Modified-Keap1

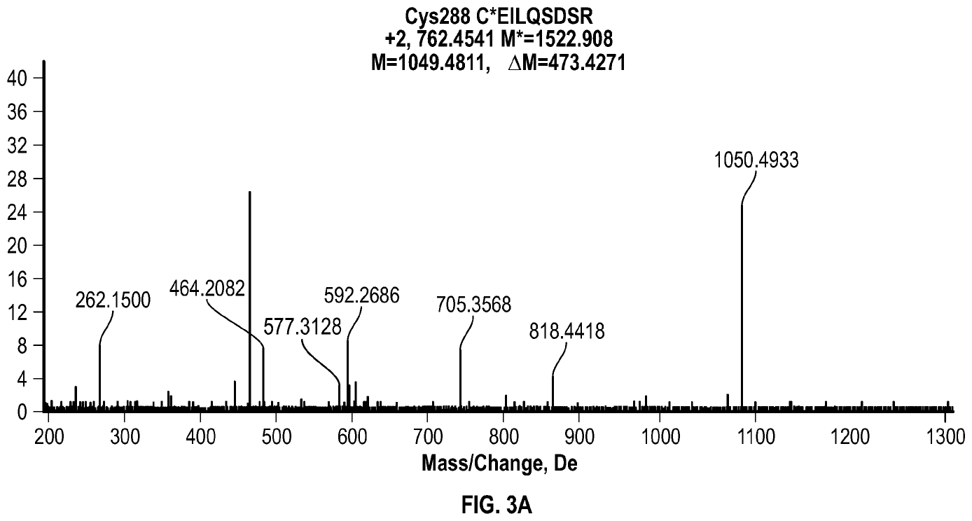

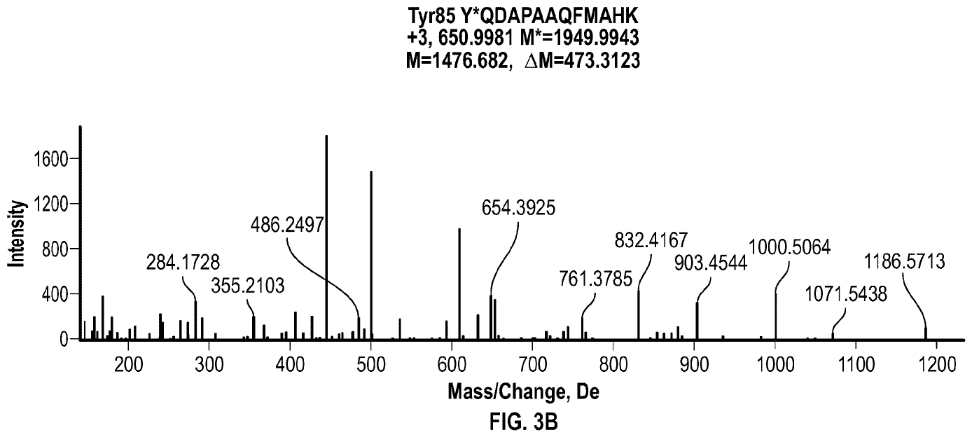

LC-MS/MS analysis of the tryptic digests of CDDO-2P-Im treated recombinant Keap1 protein revealed multiple types of adducts, including a Michael adduct formed with cysteine and acylation adducts formed with tyrosine and lysine (Table 3). The Michael addition of CDDO-2P-Im to Cys288 followed by acylation to the N-terminus amino group resulted in an adduct with a mass addition of 473.4 amu. FIG. 3A shows a MS/MS spectrum of a doubly charged ion at m/z 762.454, corresponding to the tryptic peptide CELLQSDSR with an additional mass of 473.4 amu. The peptide sequence was confirmed by partial singly charged y and b series ions. The modification site was confirmed by the presence of b1* (m/z 577.3128). The presence of a fragment ion of m/z 446.307 derived from CDDO-2P-Im provided further evidence of the modification. This adduct was also detected at Cys38 and Cys257; however, modification of Cys151 that was reported previously was not detected. Recently, modification of Cys257 and Cys288 on the protein Keap1 by an endogenous metabolite has enabled Nrf2 to increase the expression of downstream genes with antioxidant and anti-inflammatory capacities (Mills et al, Nature 556, 113-117, 2018). Our finding that CDDO-2P-Im alkylated these key cysteine residues on the Keap1 may provide therapeutic opportunities to use CDDO-2P-im to treat inflammatory diseases. Interestingly, acylation on multiple amino acids including tyrosine and lysine residues were also detected when Keap1 was treated with 100 μM CDDO-2P-Im. Although it is not clear whether modification at these sites on the Keap1 protein affects its activity, our finding provided novel mechanisms how bifunctional triterpenoids interact with the Keap1 protein.

CDDO-2P-Im covalently bound to human Keap1 protein in vitro. A total of 8 binding sites were identified when CDDO-2P-Im was incubated with Keap1 protein at the concentration of 100 μM.

TABLE 3
CDDO-2P-Im modified Keap1 peptides detected in
vitro^{a}

| Amino | Peptide sequence of | | |
|---|---|---|---|
| acid | SEQ ID NO: 3^{b} | | m/z |
| Cys38 | 34-39 | ASTEC*K | 556.1458 |
| Tyr85 | 85-97 | Y*QDAPAAQFMAHK | 651.9981 |
| Tyr208 | 205-216 | EYIY*MHFGEVAK | 654.0048 |
| Cys257 | 255-260 | YDC*EQR | 643.801 |
| Tyr263 | 262-269 | FY*VQALLR | 742.4025 |
| Cys288 | 288-296 | C*EILQSDSR | 762.4541 |
| Lys323 | 321-326 | APK*VGR | 550.864 |
| Tyr443 | 443-447 | Y*EPER | 583.812 |

^{a}CDDO-2P-Im (100 μM) was incubated with Keap1 in phosphate buffer, pH 7.4 for 16 h.
^{b}The modified amino acid was labelled with *.

### Example 4. Triterpenoid Modified-LONP1

LONP plays a crucial role in maintaining the mitochondria functions, and LONP expression levels has been associated with many human diseases. Bifunctional triterpenoids inhibit LONP, providing therapeutic opportunities to use them to treat cancer. To probe the molecular and chemical mechanisms underlying the LONP inhibition caused by triterpenoids, LONP was incubated with CDDO-2p-Im at 100 μM. LC-MS/MS analysis of the tryptic digests revealed 8 CDDO-2p-Im modified peptides, including peptides containing lysine (3), cysteine (3), and tyrosine (2) residues (Table 4).

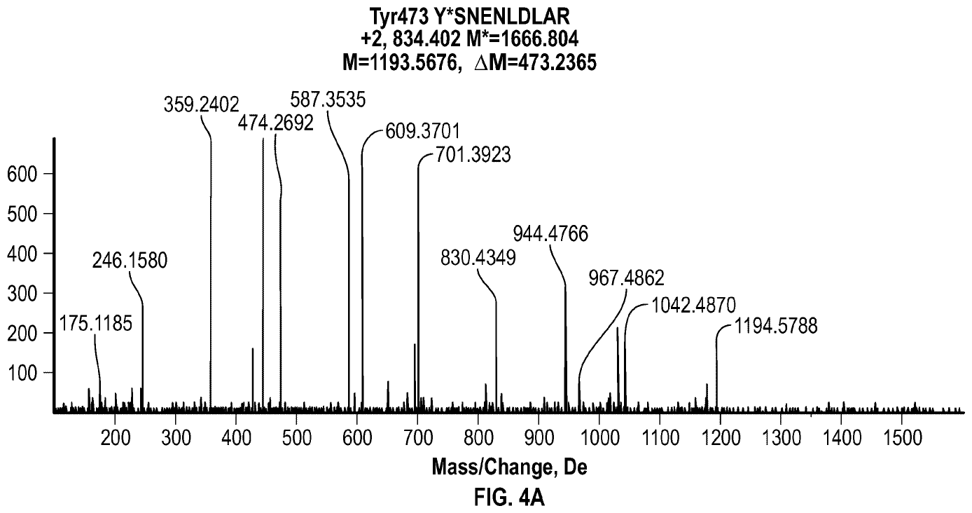

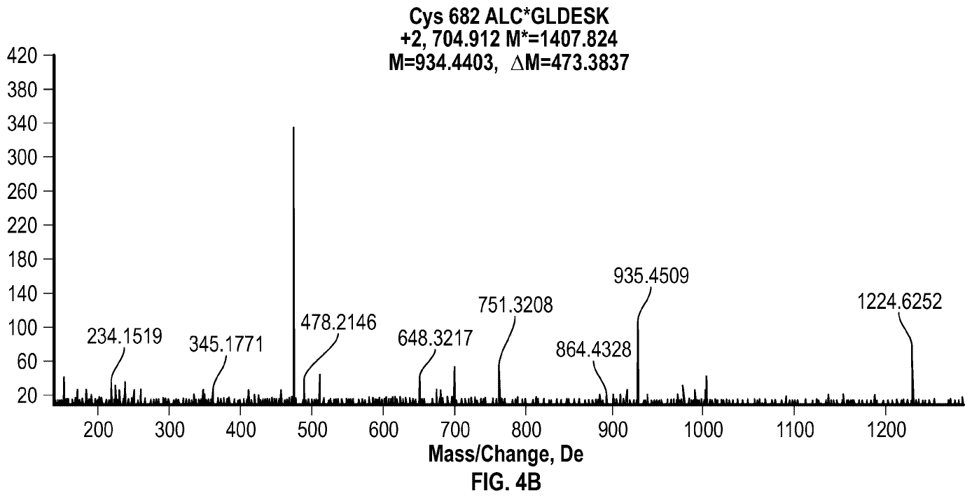

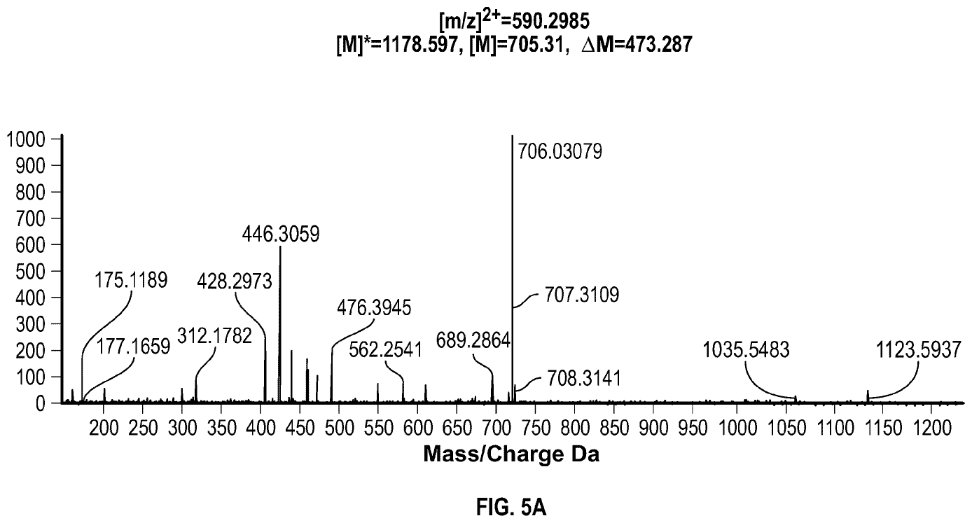

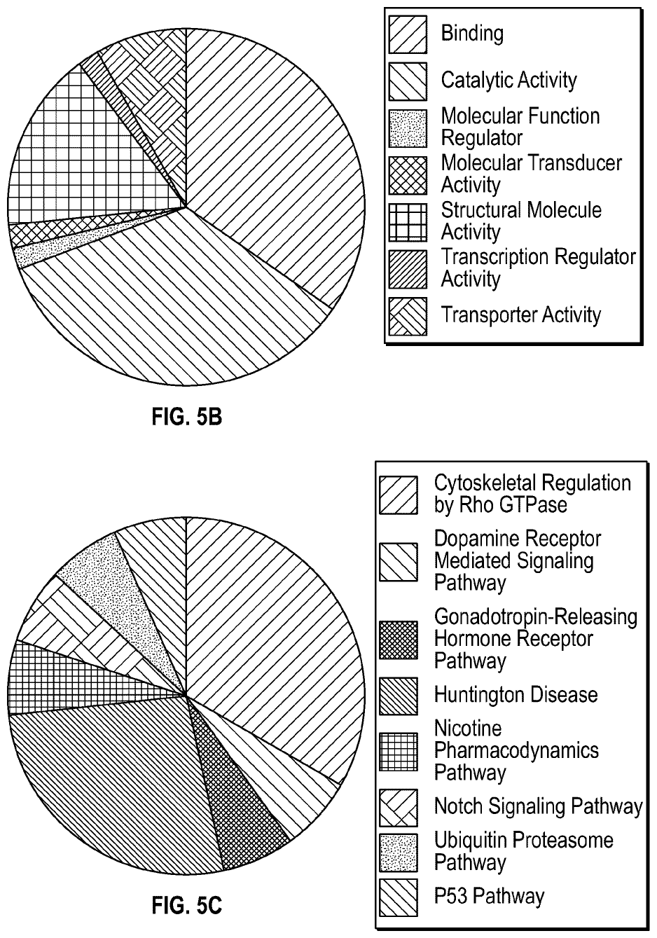

A typical MS/MS spectrum representing the tryptic peptide YSNENLDLAR with a mass addition of 473.3 amu is shown in FIG. 4A. The presence of tyrosine immonium ion (m/z 609.37) provided firm evidence of acylation at Tyr473. Similar to the cysteine modifications observed on the Keap1 protein, 3 cysteine residues (Cys520, Cys637, and Cys682) on the LONP were modified by CDDO-2P-Im through Michael addiction (FIG. 4B). Cys520 is close to the ATP binding site (523-530); covalent modification of Cys520 by CDDO-2P-Im may prevent ATP binding and therefore inhibit LONP protease activity. In addition, modification of Lys896, which is close to one of the LONP active sites (Ser855 and Lys898) may also demolish LONP protease activity.

CDDO-2P-Im covalently bound to human LONP protein in vitro.

TABLE 4
CDDO-2P-Im modified LONP peptides detected in
vitro^{a}

| Amino | Peptide sequence of | | |
|---|---|---|---|
| acid | SEQ ID NO: 4^{b} | | m/z |
| Lys426 | 425-432 | LK*ELVVPK | 700.002 |
| Tyr473 | 473-482 | Y*SNENLDLAR | 834.402 |
| Cys520 | 518-529 | ILC*FYGPPGVGK | 862.512 |
| Cys637 | 633-652 | VLFIC*TANVTDTIPEPLRDR | 916.201 |
| Tyr673 | 673-679 | Y*LVPQAR | 660.411 |
| Cys682 | 680-688 | ALC*GLDESK | 704.912 |
| Lys718 | 715-721 | QVEK*VLR | 672.914 |
| Lys896 | 889-898 | ILPVGGIK*EK | 764.001 |

aCDDO-2P-Im (100 μM) was incubated with Keap1 in phosphate buffer, pH 7.4 for 16 h.
^{b}The modified amino acid was labelled with *.

### Example 5. Additional Triterpenoid Modified-Intracellular Proteins

Human B cells were treated with triterpenoids for 16 h to examine the effects of these compounds on the cellular functions. Treatment of cells with triterpenoid inhibited B cell proliferation. To analyze the underlying mechanisms and identify downstream mediators, a proteomics analysis was initiated to identify intracellular proteins that are targeted by triterpenoids. LC-MS/MS analysis of the tryptic digests of CDDO-2P-Im treated B cells revealed multiple proteins can be targets by CDDO-2P-Im.

A total of 32 proteins were identified when B cells were treated with 10 μM CDDO-2P-Im for 16 hours (Table 5), in which Peptidylprolyl isomerase A (PPIA) appeared to be a highly specific target for CDDO-2P-Im. PPIA contains 4 active cysteine residues, all modified by CDDO-2P-Im (FIGS. 3A, B and C). PPIA catalyzes the cis-trans isomerization of proline imidic peptide bonds and accelerate protein folding. It plays an important role in regulating many biological processes including intracellular signaling, protein transport, transcription, inflammation, and apoptosis. PPIA is highly involved in acute and chronic inflammatory diseases. Thus, selective inhibition of PPIA may provide effective therapeutics for such inflammatory diseases.

In order to determine the cellular processes, molecular functions, and pathways that CDDO-2P-Im modified proteins are involved, those 32 modified proteins were analyzed by PANTHER (Protein Analysis Through Evolutionary Relationships, version 14.2). Molecular function analysis revealed that CCDO-2P-Im preferentially targeted proteins involved in binding, catalytic activity, and structural molecule activity (FIG. 3D). In addition, pathway analysis indicated that cytoskeletal regulation by Rho GTPase and Huntington disease were among the most significant pathways associated with CDDO-2P-Im modified proteins (FIG. 3E).

CDDO-2P-Im covalently bound to multiple cellular proteins in human B cells.

TABLE 5
CDDO-2P-Im modified B cell proteins detected in vitro^{a}

| Accession | SEQ | | | | |
|---|---|---|---|---|---|
| No./Name | ID No. | Position | Peptide Sequence^{b} | m/z | RT |
| P62937\|PPIA | 5 | 50-55 | GSC*FHR | 590.299 | 40.66 |
| | 5 | 50-55 | Ac-GSC*FHR | 611.803 | 43.84 |
| | 5 | 56-69 | IIPGFMC*QGGDFTR | 672.344 | 46.93 |
| | 5 | 156-165 | ITIADC*GQLE | 768.405 | 47.99 |
| | 5 | 92-118 | HTGPGILSMANAGPNTNGSQFFIC*TAK | 802.656 | 44.56 |
| | 5 | 155-165 | KITLADC*GQLE | 832.449 | 44.85 |
| O00299\|CLIC1 | 6 | 21-29 | IGNC*PFSQR | 747.89 | 45.02 |
| Q96NY7\|CLIC6 | 7 | 484-492 | | | |
| O14841\|OPLA | 8 | 497-501 | C*AIAR | 503.789 | 42.1 |
| O60423\|AT8B3 | 9 | 865-871 | RLSLLC*R | 675.908 | 45.66 |
| P05023\|AT1A1 | 10 | 424-430 | IAGLC*NR | 610.343 | 44.73 |
| P07437\|TBB5 | 11 | 298-306 | NMMAAC*DPR | 741.351 | 44.83 |
| P07737\|PROF1 | 12 | 117-136 | EGVHGGLINKKC*YEMASHLR | 543.891 | 36.21 |
| P08865\|RSSA | 13 | 156-166 | YVDIAIPC*NNK | 861.954 | 44.89 |
| P09382\|LEG1 | 14 | 50-74 | FNAHGDANTIVC*NSKDGGAWGTEQR | 781.126 | 36.97 |
| | 14 | 2-21 | Ac-ACGLVASNLNLKPGEC*LRVR | 810.768 | 48.6 |
| | 14 | 3-21 | CGLVASNLNLKPGEC*LRVR | 821.427 | 48.74 |
| P09429\|HMGB1 | 15 | 30-48 | KHPDASVNFSEFSKKC*SER | 534.676 | 35.27 |
| | 15 | 31-48 | HPDASVNFSEFSKKC*SER | 636.07 | 36.76 |
| P09651\|ROA1 | 16 | 167-179 | YHTVNGHNC*EVRK | 508.264 | 32.51 |
| | 16 | 167-178 | YHTVNGHNC*EVR | 476.241 | 35.52 |
| P22314\|UBA1 | 17 | 628-635 | SIPIC*TLK | 674.395 | 47.15 |
| P23528\|COF1 | 18 | 35-45 | AVLFC*LSEDKK | 575.986 | 44.43 |
| | 18 | 128-146 | LTGIKHELQANC*YEEVKDR | 680.612 | 35.83 |
| P31146\|COR1A | 19 | 13-29 | HVFGQPAKADQC*YEDVR | 609.808 | 37.15 |
| P43243\|MATR3 | 20 | 170-192 | C*RDDSFFGETSHNYHKFDSEYER | 669.305 | 35.24 |
| P46782\|RS5 | 21 | 64-71 | AQC*PIVER | 694.883 | 42.88 |
| P52790\|HXK3 | 22 | 99-104 | TGAS*LR | 539.296 | 41.92 |
| P54577\|SYYC | 23 | 514-520 | LGSISC*K | 590.834 | 42.92 |
| P61978\|HNRPK | 24 | 140-148 | GSDFDC*ELR | 757.863 | 44.23 |
| P63220\|RS21 | 25 | 16-22 | KC*SASNR | 413.555 | 35.62 |
| P68363\|TBA1B | 26 | 339-352 | RSIQFVDWC*PTGFK | 719.714 | 46.18 |
| | 26 | 340-352 | SIQFVDWC*PTGFK | 1001.01 | 49.13 |
| P84103\|SRSF3 | 27 | 70-75 | TLCGC*R | 591.804 | 43.9 |
| Q00839\|HNRPU | 28 | 603-609 | AVVVC*PK | 594.854 | 43.94 |
| Q06830\|PRDX1 | 29 | 169-190 | HGEVC*PAGWKPGSDTIKPDVQK | 706.875 | 35.87 |
| Q12802\|AKP13 | 30 | 1548-1552 | C*SVLR | 525.804 | 42.59 |
| Q15365\|PCBP1 | 31 | 102-115 | LVVPATQC*GSLIGK | 930.041 | 46.29 |
| Q5IR90\|Q5IR90 | 32 | 245-250 | C*PLVLK | 573.35 | 46.07 |
| Q5T890\|ER6L2 | 33 | 804-808 | C*GFSK | 507.767 | 43.16 |
| Q92945\|FUBP2 | 34 | 436-442 | C*GLVIGR | 595.852 | 45.83 |
| Q9BVA1\|TBB2B | 35 | 351-359 | TAVC*DIPPR | 722.896 | 44.13 |

^{a}CDDO-2P-Im (100 μM) was incubated with B cells for 16 h.
^{b}The modified amino acid was labelled with *.

1. A method for irreversibly inhibiting the activity of a protein in a patient or in a biological sample comprising the step of administering to said patient or contacting said biological sample with 1-[2-Cyano-3,12-dioxooleana-1,9(11)-dien-28-oyl]-4(-pyridin-2-yl)-1H-imidazole (CDDO-2P-Im) or a pharmaceutically acceptable salt thereof or 1-[2-Cyano-3,12-dioxooleana-1,9(11)-dien-28-oyl]-4(-pyridin-3-yl)-1H-imidazole (CDDO-3P-Im) or a pharmaceutically acceptable salt thereof, wherein the activity of the protein is irreversibly inhibited by covalently modifying a nucleophilic amino acid residue contained therein.

2. The method of claim 1, wherein the nucleophilic amino acid residue is a lysine, serine, arginine, or tyrosine residue.

3. The method of claim 1, wherein the protein is human GSTP, human LONP1, human Keap1, or human PPIA.

4. The method of claim 1, wherein the protein is human GSTP.

5. The method of claim 1, wherein the protein is human GSTP and the nucleophilic amino acid residue is Cys15, Cys48, Cys102, or Arg187 of SEQ ID NO: 1.

6. The method of claim 5, wherein the compound is CDDO-2P-Im or a pharmaceutically acceptable salt thereof.

7. The method of claim 5, wherein the compound is CDDO-3P-Im or a pharmaceutically acceptable salt thereof.

8. The method of claim 1, wherein the protein is human LONP1.

9. The method of claim 1, wherein the protein is human LONP1 and the nucleophilic amino acid residue is Lys426, Tyr473, Cys520, Cys637, Tyr673, Cys682, Lys718, or Lys896 of SEQ ID NO: 4.

10. The method of claim 9, wherein the compound is CDDO-2P-Im or a pharmaceutically acceptable salt thereof.

11. The method of claim 9, wherein the compound is CDDO-3P-Im or a pharmaceutically acceptable salt thereof.

12. The method of claim 1, wherein the protein is human Keap1.

13. The method of claim 1, wherein the protein is human Keap1 and the nucleophilic amino acid residue is Cys38, Tyr85, Tyr208, Cys257, Tyr263, Cys288, Lys323, or Tyr443 of SEQ ID NO: 3.

14. The method of claim 13, wherein the compound is CDDO-2P-Im or a pharmaceutically acceptable salt thereof.

15. The method of claim 13, wherein the compound is CDDO-3P-Im or a pharmaceutically acceptable salt thereof.

16. The method of claim 1, wherein the protein is human PPIA.

17. The method of claim 1, wherein the protein is human PPIA and the nucleophilic amino acid residue is Cys52, Cys62, Cys115, or Cys161 of SEQ ID NO: 5.

18. The method of claim 17, wherein the compound is CDDO-2P-Im or a pharmaceutically acceptable salt thereof.

19. The method of claim 17, wherein the compound is CDDO-3P-Im or a pharmaceutically acceptable salt thereof.

20. The method of claim 1, wherein the method comprises the step of administering CDDO-2P-Im or a pharmaceutically acceptable salt thereof or CDDO-3P-Im or a pharmaceutically acceptable salt thereof to said patient, wherein said patient is a brain cancer patient or a patient having a neurodegenerative or neuropsychiatric disease.
